(19) United States
(12) Patent Application Publication
(10) Pub. No.: US 2015/0044135 A1
(43) Pub. Date: Feb. 12, 2015

(54) DUAL FUNCTION MARKERS FOR DIAGNOSTICS AND THERAPEUTICS FOR UPPER GASTROINTESTINAL TRACT PRECANCER

(71) Applicants: Wa Xian, Immunos (SG); Frank McKeon, Cambridge, MA (US); Matthew Vincent, Newburyport, MA (US); Khek Yu Ho, Singapore (SG)

(72) Inventors: Wa Xian, Immunos (SG); Frank McKeon, Cambridge, MA (US); Matthew Vincent, Newburyport, MA (US); Khek Yu Ho, Singapore (SG)

(21) Appl. No.: 14/494,799

(22) Filed: Sep. 24, 2014

Related U.S. Application Data
(63) Continuation of application No. PCT/US2013/034325, filed on Mar. 28, 2013.
(60) Provisional application No. 61/618,668, filed on Mar. 30, 2012.

(51) Int. Cl. C12Q 1/68; C07K 16/30; G01N 33/50; G01N 33/574

(52) U.S. Cl. CPC C12Q 1/6886; G01N 33/57446; G01N 33/57492; C07K 16/3046; G01N 33/5011; C12Q 2600/158; G01N 2333/705
USPC 424/1.49; 435/325; 435/6.13; 435/7.23; 435/366; 424/9.34; 424/9.4; 424/9.6; 506/9; 424/174.1; 424/143.1; 424/178.1; 514/44 A; 530/391.3; 435/32

(57) ABSTRACT

The invention described herein relates to the detection, diagnosis, and treatment of intestinal metaplasias that develop to esophageal, gastric, and pancreatic adenocarcinoma. The stem cells and differentiated cells of these intestinal metaplasias show high expression of CDH17 as well as other proteins. The invention also includes a clonal population of Barrett's esophagus stem cells as well as the stem cells of the surrounding normal epithelia and methods of using them for the detection, diagnosis, and treatment of Barrett's esophagus.

### RELATED APPLICATION

This application claims priority to U.S. Provisional Application No. 61/618,668 filed Mar. 30, 2012, the content of which are hereby incorporated herein in its entirety.

### GOVERNMENTAL FUNDING

The invention described herein was supported, in part, by grants from the National Institutes of Health (R01GM 083348). The United States government may have certain rights in the invention.

### FIELD OF THE INVENTION

The invention described herein relates to the detection, diagnosis, and treatment of precursors of various cancers, including those of esophageal, gastric, and pancreatic adenocarcinoma.

### BACKGROUND OF THE INVENTION

Esophageal and gastric adenocarcinoma together kill more than one million people each year worldwide and represent the 2nd leading cause of death from cancer. Both cancers arise in association with chronic inflammation and are preceded by robust metaplasia with intestinal characteristics. In fact, the patient population with precancerous lesions is estimated to be significantly larger—in the range of 100 million people in size—all at substantial risk of developing cancer in their lifetimes. Current treatments for both cancer and precancerous patients have an exceptionally high degree of relapse, with the 5 year survival rate for patients developing cancer being marginal.

Gastric intestinal metaplasia can be triggered by gastritis involving H. pylori infections, while Barrett's metaplasia of the esophagus is linked to gastroesophageal reflux disease (GERD). While H. pylori suppression therapies have contributed to the recent decline of gastric adenocarcinoma, the incidence of esophageal adenocarcinoma, especially in the West, has increased dramatically in the past several decades. (Spechler et al. N Engl J Med. 1986; 315:362-71; Blot et al. JAMA 1991; 265:1287-9; Raskin et al. Cancer Res 1992; 52:2946-50; Jankowski et al. Am Pathol 1999; 154:965-973; and Reid et al. Nat Rev Cancer 2010; 10:87-101). Treatments for late stages of these diseases are challenging and largely palliative and therefore considerable efforts have focused on understanding the earlier, premalignant stages of these diseases for therapeutic opportunities.

The prevailing theory for the development of metaplasia has been that the abnormal cells seen in Barrett's esophagus arise as the normal squamous cells “transcommit” in response to inflammation (such as acid-reflux) to a new, intestine-like fate. Intestine-like metaplasia is a columnar epithelium marked by prominent goblet cells and intestinal markers such as villin and trefoil factors 1, 2, and 3, and, once established, appears to be irreversible (Sagar et al. Br J Surg. 1995; 82:806-10; Barr et al. Lancet 1996; 348:584-5; and Watari et al. Clin Gastroenterol Hepatol 2008; 6:409-17). There is compelling evidence for a dynamic competition among clones of cells within Barrett's metaplasia that almost certainly contributes to its premalignant progression. Cancers arise from this metaplasia via stereotypic genetic and cytological changes that present as dysplasia, high-grade dysplasia, and finally invasive adenocarcinoma (Raskin et al., supra; Jankowski et al., supra; Haggitt. Hum Pathol 1994; 25:982-93; Schlemper et al. Gut 2000; 47:251-5; and Correa et al. Am J Gastroenterol 2010; 105:493-8).

### SUMMARY OF THE INVENTION

An understanding of the ontogeny of gastric intestinal metaplasia would allow for the development of compositions and methods for the early detection and treatment of gastric intestinal metaplasia prior to progression to adenocarcinoma. As described in greater detail herein, the inventors have replaced the old paradigm of transcommittment of cell fate with a new understanding of the origins of esophageal and gastric metaplasias in which stem cells of embryonic origin—left behind during organogenesis of the alimentary canal—give rise to the precancerous diseases and ultimately to esophageal and gastric adenocarcinoma. The inventors have shown that this discrete population of stem cells persists in humans at the squamocolumnar junction, the source of Barrett's metaplasia. The inventors have also shown that upon damage to the squamous epithelium, these stem cells are activated and proliferate in the development of the precancerous lesions. The findings presented in this application demonstrate that gastric intestinal and Barrett's metaplasias initiate not from genetic alterations or transcommittment of differentiated tissue, but rather from competitive interactions between cell lineages driven by opportunity. Targeting these precancerous lesions by preventing growth and/or differentiation of these vestigial stem cells, which have proven to be resistant to physical ablation and other therapies directed to the resulting metaplasias, offers a unique opportunity to prevent progression to cancer in a very large patient population.

As described in further detail in this application, the inventors have isolated these stem cells from these cancer precursors, as well as normal epithelial stem cells for the esophagus, stomach and intestines, and through gene expression profiling have identified a number of targets for development of antibodies, RNAi and small molecule therapeutics that may be selectively lethal to the stem cells in these cancer precursors (intestinal metaplasia) relative to nearby regions of the alimentary canal. With the isolated cells in hand, there is now the opportunity to rapidly develop drug candidates with selectivity and in vitro efficacy. Coupled with animal models for these diseases presented herein and others available in the art, there is a clear preclinical and clinical path to providing effective therapies. While it is expected that systemic delivery of therapeutic agents is an option, the fact of the matter is that the sites of treatment lend themselves well to oral or endoscopic depot delivery. The dim prognosis for esophageal, gastric and pancreatic adenocarcinoma argues for therapies directed at preventing even the initiation of the precancerous metaplasia. For these precancerous metaplasia patients again numbering in the tens of millions—this provides a ten to twenty year window for treatment before cancer would typically develop.

Accordingly, a salient feature to the current application is the discovery that a unique population of primitive epithelial stem cells give rise to the metaplasia underlying esophageal, gastric, and pancreatic adenocarcinoma and that these primitive epithelial stem cells have a distinct molecular signature that can be exploited for diagnostic and therapeutic targeting. For instance, these discoveries allow for the therapeutic targeting of the population of stem cells responsible for the metaplasia using cytotoxic and/or growth inhibitory and/or differentiation inhibitory agents, particularly agents selective for the stem cell relative to normal squamous cells or regenerative stem cells of the esophagus or stomach, thus facilitating the treatment of metaplasia and prevention of its progression to adenocarcinoma. Likewise, the use of agents directed to gene products unique to the stem cell, particularly cell surface markers that can be detected with antibodies, the present invention provides reagents and methods for detecting the stem cell in tissue biopsy samples as well as in vivo (i.e., for imaging or detection using endoscopic visualization). Given the accessibility of these tissues through non-invasive and minimally invasive techniques, in certain preferred embodiments the therapeutic agents or imaging agents are delivered by direct application or injection, such as by endoscopic.

The following are merely illustrative. In the case of a gene encoding a cell surface protein, the therapeutic agent can be an antibody or antibody mimetic, i.e., one which inhibits growth or differentiation by inhibiting the function of the cell surface protein, or one which is cytotoxic to the cell as a consequence to invoking an immunological response (i.e., ADCC) against the targeted stem cell. In the case of a gene encoding an enzyme, the therapeutic may be a small molecule inhibitor of the enzymatic activity, or a prodrug including a substrate for the enzyme such that the prodrug is converted to an active agent upon cleavage of the substrate portion. In the case of transcription factors, the therapeutic agent may be a decoy nucleic acid that competes with the genomic regulatory elements for binding to the transcription factor; or in the case of ligand-regulated transcription factors, may be an agonist or antagonist ligand of the transcription factor. In instances where the viability, growth or differentiation of the target stem cell is dependent on the level of expression of the gene, then use of antisense, RNAi or other inhibitory nucleic acid therapeutics can be considered.

In one aspect, the invention provides a method for treating or preventing esophageal metaplasia, comprising administering to a subject a therapeutic amount of an agent that decreases the expression and/or biological activity of one or more of the genes set forth in Table 1, such that the metaplasia is treated or prevented. In certain embodiments, the agent is an antibody, antibody-like molecule, antisense oligonucleotide, small molecule or RNAi agent.

In another aspect, the invention provides a method for treating or preventing esophageal metaplasia, comprising administering a therapeutic amount of an agent that specifically binds to or reduces the expression of a polypeptide encoded by one of the genes set forth in Table 1, wherein said agent is linked to one or more cytotoxic moiety. In one embodiment, the gene is CDH17. In certain embodiments, the agent is an antibody, antibody-like molecule or cell surface receptor ligand. The cytotoxic moiety can be, for example, a radioactive isotope, chemotoxin, or toxin protein. In certain embodiments, the cytotoxic moiety is encapsulated in a biocompatible delivery vehicle including, without limitation, microcapsules, microparticles, nanoparticles, and liposomes. In some embodiments, the agent is directly linked to the cytotoxic moiety.

In another aspect, the invention provides a method of imaging esophageal metaplasia, the method comprising administering to a subject an effective amount of an agent that specifically binds to or reduces the expression of a polypeptide encoded by one of the genes set forth in Table 1, and a visualizing the agent. In certain embodiments, the agent is an antibody, antibody-like molecule or cell surface receptor ligand. In certain embodiments, the agent is linked to an imaging moiety. The imaging moiety can be, for example, a positron-emitter, nuclear magnetic resonance spin probe, an optically visible dye, or an optically visible particle. The imaging agent may be one that permits non-invasive imaging, such as by MRI, PET or the like. In other embodiments, the imaging moiety can be a fluorescent probe or other optically active probe which can be visualized, e.g., through an endoscope.

According to the methods of the invention, a therapeutic and/or imaging agent can be administered by any suitable route and/or means including, without limitation, orally and/or parenterally. In a preferred embodiment, the agent is administered endoscopically to the esophageal squamocolumnar junction or a site of esophageal metaplasia.

In another aspect, the invention provides a method of detecting the presence or absence of the target stem cell in a tissue biopsy. Such detection agents can include antibodies and nucleic acids which bind to a gene or gene product unique to the stem cell relative to other normal or diseased esophageal tissue.

In another aspect, the invention provides a method of diagnosing, or predicting the future development or risk of development of, esophageal metaplasia or adenocarcinoma, comprising measuring the expression level of one or more of the genes set forth in Table 1 in an epithelial tissue sample from a subject, wherein an increase in the expression level relative to a suitable control indicates that the subject has, or has a future risk of developing, metaplasia. In one embodiment, the gene is CDH17. In some embodiments, mRNA levels of the gene are measured. In other embodiments, the levels of the protein product of the gene are measured. Such methods can be performed in vivo or in vitro.

The invention further provides a composition comprising a clonal population of Barrett's Esophagus (BE) stem cells, such as may be isolated from an esophagus of a subject or generated from ES cells or iPS cells, wherein the stem cells differentiate into Barrett's epithelium (i.e., columnar epithelium). Preferably the composition, with respect to the cellular component, is at least 50 percent BE stem cell, more preferably at least 75, 80, 85, 90, 95 or even 99 percent BE stem cell. The BE stem cells can be pluripotent, multipotent or oligopotent. In certain preferred embodiments, the BE stem cells are characterized as having an mRNA profile can further include a profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA in the clonal cell population are each in the range of 5 to 50 percent of the amount of actin mRNA in the clonal cell population, more preferably in the range of 10-25 percent. Preferably all fourteen genes have an mRNA profile in that range. In one embodiment, CDH17 mRNA is in the range of 5 to 50 or 10-25 percent of the amount of actin mRNA. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA.

In an additional embodiment, the BE stem cells are characterized as CDH17-positive, and Krt20-, Sox2- and p63-negative, as detected by standard antibody staining. For instance, levels of Krt20, Sox2 and p63 are less than 10 percent of the level of CDH17, and more preferably less than 5 percent, 1 percent, and even less than 0.1 percent.

The invention further provides a composition comprising a population of cells enriched in a clonal subpopulation of BE stem cells from an esophagus of a subject, wherein the clonal subpopulation of cells differentiates into Barrett's epithelium (i.e., columnar epithelium). The BE stem cells can be pluripotent, multipotent or oligopotent.

Another aspect of the invention provides a clonal population of Barrett's Esophagus (BE) stem cells, derived from patients or iPS cell sources, characterized as having an mRNA profile can further include a profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA in the stem cell population are each in the range of 5 to 50 percent of the amount of actin mRNA in the clonal cell population, more preferably in the range of 10-25 percent. Preferably all fourteen genes have an mRNA profile in that range. In one embodiment, CDH17 mRNA is in the range of 5 to 50 or 10-25 percent of the amount of actin mRNA. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The clonal population of BE stem cells may also be characterized as CDH17positive, and Krt20, Sox2 and p63 negative, as detected by standard antibody staining. For instance, levels of Krt20, Sox2 and p63 are less than 10 percent of the level of CDH17, and more preferably less than 5 percent, 1 percent, and even less than 0.1 percent.

The invention further provides a method of screening for an agent effective in the treatment or prevention of Barrett's esophagus including the steps of providing a population of BE stem cells, wherein the BE stem cells are able to differentiate into Barrett's epithelium; providing a test agent; and exposing the BE stem cells to the test agent; wherein if the test agent is cytotoxic, cytostatic and/or able to inhibit the differentiation of the BE stem cells to columnar epithelial cells, the test agent is an agent effective in the treatment or prevention of Barrett's esophagus.

In certain embodiments, the BE stem cells are mammalian BE stem cells, such as human BE stem cells.

In certain embodiments, candidate therapeutic agents reduce the viability, growth or ability to differentiation by 70, 80, 90, 95, 96, 97, 98, 99 or even 100%.

The BE stem cells can be clonal, and can be pluripotent, multipotent or oligopotent. In certain preferred embodiments, the BE stem cells are characterized as having an mRNA profile can further include a profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA in the stem cell population are each in the range of 5 to 50 percent of the amount of actin mRNA in the stem cell population, more preferably in the range of 10-25 percent. Preferably all fourteen genes have an mRNA profile in that range. In one embodiment, CDH17 mRNA is in the range of 5 to 50 or 10-25 percent of the amount of actin mRNA. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The clonal population of BE stem cells may also be characterized as CDH17-positive, and Krt20-, Sox2- and p63-negative, as detected by standard antibody staining. For instance, levels of Krt20, Sox2 and p63 are less than 10 percent of the level of CDH17, and more preferably less than 5 percent, 1 percent, and even less than 0.1 percent.

The invention further provides a method of screening for an agent effective in the detection of Barrett's esophagus including the steps of providing BE stem cells; providing a test agent; and exposing the BE stem cells to the test agent; wherein if the test agent specifically binds to the BE stem cells, i.e., relative to normal squamous cells or intestinal cells or gastric epithelial cells, the test agent is an agent effective in the detection of stem cells giving rise to Barrett's esophagus. In certain embodiments, the test agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4. In one embodiment, the test agent specifically binds to CDH17.

In certain embodiments, the BE stem cells are mammalian, and more preferably are human.

The invention further provides a method of detecting the presence of Barrett's esophagus in a subject including the steps of providing a detection agent that specifically binds to BE stem cells and BE differentiated cells; administering the detection agent to a subject; and detecting whether the detection agent specifically binds to BE cells in the esophagus of the subject, wherein, if the detection agent specifically binds to a cell in the esophagus of the subject to a higher degree than the average non-Barrett's esophagus patient, the subject is diagnosed with Barrett's esophagus or as having a risk of developing Barrett's esophagus. In certain embodiments, the detection agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4. In one embodiment, the detection agent specifically binds to CDH17.

The invention further provides a method of for treating or preventing Barrett's esophagus and/or esophageal metaplasia in a subject in need thereof comprising administering to subject an effective amount of an agent that is cytotoxic or cytostatic for Barrett's esophagus stem cells in the esophagus of the subject, or inhibits differentiation of the Barrett's esophagus stem cells to columnar epithelium. In certain embodiments, the agent specifically binds to or reduces the expression of one or more of CDH17, CLRN3, TM4SF4, FAM3B, NMUR2, MUC17, CEACAM7, ANXA13, SLC16A4, CD44, NPNT, PLBD1, TFF3 and REG4. In one embodiment, the agent specifically binds to or reduces the expression of CDH17.

In certain embodiments, the subject is a mammal. In a preferred embodiment, the mammal is human.

In certain embodiments, candidate therapeutic agents reduce the viability, growth or ability to differentiation by 70, 80, 90, 95, 96, 97, 98, 99 or even 100%.

The targeted BE stem cells can characterized as having an mRNA profile that can further include a profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA in the stem cell population are each in the range of 5 to 50 percent of the amount of actin mRNA in the stem cell population, more preferably in the range of 10-25 percent. Preferably all fourteen genes have an mRNA profile in that range. In one embodiment, CDH17 mRNA is in the range of 5 to 50 or 10-25 percent of the amount of actin mRNA. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The stem population of BE stem cells may also be characterized as CDH17-positive, and Krt20-, Sox2- and p63-negative, as detected by standard antibody staining. For instance, levels of Krt20, Sox2 and p63 are less than 10 percent of the level of CDH17, and more preferably less than 5 percent, 1 percent, and even less than 0.1 percent.

The invention further provides a method of detecting the presence of Barrett's esophagus stem cells in a subject including the steps of providing a detection agent that specifically binds to BE stem cells; administering the detection agent to a subject; and detecting whether the detection agent specifically binds to a BE stem cell in the esophagus of the subject, wherein, if the detection agent specifically binds to a cell in the esophagus of the subject to a higher degree than the average non-Barrett's esophagus patient, the subject is diagnosed with Barrett's esophagus or as having a risk of developing Barrett's esophagus. In certain embodiments, the detection agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4. In one embodiment, the detection agent specifically binds to CDH17.

The test agent can also be an RNAi or antisense composition. The RNAi or antisense composition can reduce the amount of mRNA in the targeted BE stem cells of a member of the group consisting of CDH17, CLRN3, TM4SF4, FAM3B, NMUR2, MUC17, CEACAM7, ANXA13, SLC16A4, CD44, NPNT, PLBD1, TFF3 and REG4. In one embodiment, the RNAi or antisense composition can reduce the amount of mRNA in the targeted BE stem cells of CDH17.

The invention further provides a method of detecting the likelihood of the presence of intestinal metaplasia (IM), in a patient including the steps of providing a detection agent that specifically binds to CDH17; administering the detection agent to a patient or contacting the detection agent with a biopsy therefrom; and detecting whether the detection agent binds to the cells in the biopsy of the patient, wherein if the detection agent binds to the cells in the biopsy of the patient the gastric IM is more likely to be present in the patient. In certain embodiments, detection agents for the detection of Villin or CDX2 can also be used to confirm the presence of IM.

In one embodiment, the intestinal metaplasia includes a cell type selected from the group consisting of Barrett's esophagus stem cells, gastric adenocarcinoma precursors and pancreatic adenocarcinoma precursors. In another embodiment, the intestinal metaplasia is selected from the group consisting of Barrett's esophagus, gastric intestinal metaplasia and intraepithelial pancreatic mucinous metaplasia.

In one embodiment, the patient is a human. In another embodiment, the detection step is performed in vitro on a biopsy sample. Specifically, the detection step can be performed in vivo. The detection agent can be an antibody. More specifically, the detection agent is a monoclonal antibody.

In other embodiments, the detection agent is a Positron Emission Tomography (PET) imaging agent or a magnetic resonance imaging (MRI) contrast agent. The detection agent can be a radioisotope or contrast enhancing isotope, such as ³H, ¹¹C, ¹⁷⁷Lu, ¹¹¹Indium, ⁶⁷Cu, ^{99m}Tc, ¹²⁴I, ¹²⁵I, ¹³¹I and ⁸⁹Zr. The detection agent can also be detected in the patient by Single Photon Emission Computed Tomography (SPECT), Positron Emission Tomography (PET), Magnetic Resonance Imaging (MRI), Fluorescent Imaging, or Near-infrared (NIR) Emission Spectroscopy.

The invention also provides a composition comprising a binding agent that specifically binds to a protein selected from the group consisting of CDH17, CLRN3, TM4SF4, FAM3B, NMUR2, MUC17, CEACAM7, ANXA13, SLC16A4, CD44, NPNT, PLBD1, TFF3 and REG4 attached to an imaging agent. In one embodiment, the protein is CDH17.

In other embodiments the binding agent is covalently or non-covalently attached to the imaging agent. The imaging agent can be selected from optical coherence tomography (OCT) detection/contrast agents, positron emission tomography (PET) detection/contrast agents, magnetic resonance imaging (MRI) detection/contrast agents, ultrasound detection/contrast agents, X-ray detection/contrast agents and single-photon emission computed tomography (SPECT) detection/contrast agents.

OCT detection/contrast agents are can include near-infrared dyes, polypyrrole nanoparticles, optical detection/contrast agents and engineered microsphere contrast agents. PET detection/contrast can include ¹⁸F-fluoride, 3′-deoxy-3′-[¹⁸F]fluorothymidine, ¹⁸F-fluoromisonidazole, gallium, technetium-99m, thallium, oxygen, nitrogen, iron, carbon, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, and ⁹⁹Tc. MRI detection/contrast agents can include ferro, antiferro, ferrimagnetic or superparamagnetic material, ferrite with spinel structure, ferrite with a magnetoplumbite structure other hexagonal ferrite structures, paramagnetic ions, comprise a paramagnetic contrast agent, a super paramagnetic contrast agent, a diamagnetic agent and combinations thereof. Ultrasound detection/contrast agents can include shell encapsulated gas bubbles; shell encapsulated droplets; and nanoparticles. X-ray detection/contrast agents can include iodinated contrast-enhancing units; barium sulfate-based contrast-enhancing units; metal ion chelates; boron clusters with a high proportion of iodine; iodinated polysaccharides, polymeric triiodobenzenes; particles from iodinated compounds displaying low water solubility; liposomes containing iodinated compounds; and iodinated. SPECT detection/contrast agents can include 99mTc, ¹²³I, ¹³¹I, ⁶⁷Cu, ¹¹¹In, and ²⁰¹Tl. Binding agent can include antibodies, aptamers, peptides, cell surface receptor ligands, and small molecules.

The invention further provides a method of screening for an agent which may be used to treat or prevent the occurrence of Barrett's esophagus, including the steps of providing BE stem cells; providing one or more of esophageal and gastric cardia stem cells; contacting the BE stem cells and one or more of esophageal and gastric cardia stem cells with the test agent; and detecting the ability of the test agent to reduce viability, growth or differentiation of the BE stem cells relative to one or more of esophageal and gastric cardia stem cells; wherein if the test agent reduces the viability, growth or differentiation of the BE stem cells relative to one or more of esophageal and gastric cardia stem cells than the test agent may be effective in the treatment or prevention of Barrett's esophagus.

In one embodiment, the test agent specifically binds to a protein selected from the group consisting of and wherein the test agent specifically binds to or reduces the mRNA expression of one or more of CDH17, CLRN3, TM4SF4, FAM3B, NMUR2, MUC17, CEACAM7, ANXA13, SLC16A4, CD44, NPNT, PLBD1, TFF3 and REG4. In certain specific embodiments, the test agent specifically binds to or reduces the expression of CDH17.

In other embodiments, the test agent is also contacted with normal cells or tissue of the local alimentary canal, and the differential ability, if any, of the test agent to reduces the viability, growth or differentiation of the normal cells or tissue is compared to that with the BE stem cells. The BE stem cells can be human BE stem cells. In other embodiments, the test agent is selected for further drug development if the test agent reduces the viability, growth or ability to differentiation of the BE stem cells is reduced by at least 70%.

In certain embodiments, the BE stem cells are provided as a clonal population of cells. The test agent can be a small molecule, carbohydrate, peptide or nucleic acid. The test agent can specifically bind to a cell surface protein on the clonal population of cells. The test agent can an antibody or antibody mimetic.

The invention further provides a method of screening for an agent effective in the detection of Barrett's esophagus including the steps of providing a BE stem cells; providing one or more of esophageal and gastric cardia stem cells; contacting the BE stem cells and one or more of esophageal and gastric cardia stem cells with the test agent; and detecting the ability of the test agent to bind to the BE stem cells and one or more of esophageal and gastric cardia stem cells; wherein if the test agent binds to the BE stem cells with greater affinity than it binds to one or more of esophageal and gastric cardia stem cells, the test agent may be an agent effective in the detection of Barrett's esophagus.

In one embodiment, the test agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4. In certain specific embodiments, the test agent specifically binds to CDH17.

In certain embodiments, the BE stem cells are human BE stem cells. The BE stem cells can be provided as a clonal population of cells. In certain embodiments, the test agent is also contacted with normal cells or tissue of the alimentary canal, and the differential ability, if any, of the test agent to bind to the normal cells or tissue is compared to that with the BE stem cells. The test agent can be an antibody or antibody mimetic. Also, the test agent can be a monoclonal antibody.

### BRIEF DESCRIPTION OF THE FIGURES

FIG. 1. Immunostaining from the gastroesophageal junction for E-cadherin, p63 and Sox2 on Barrett's, esophageal and gastric cardia stem cells. Both esophageal and gastric cardia stem cells were SOX2 positive, which is consistent with previous published findings. Importantly, the Barrett's stem cells are SOX2- and p63-, suggesting that it is a unique population of the cells at the gastroesophageal (GE) junction

FIG. 2. Heat map of gene expression of Barrett's, esophageal and gastric cardia stem cells found in the gastroesophageal junction.

FIG. 3. Immunostaining and immunohistochemistry showing CDH17 expression in Barrett's stem cells in culture and Barrett's tissue in tissue sections but not esophageal or gastric cardia stem cells or tissue.

FIG. 4. Immunohistochemistry showing Cdx2 and Cdh17 expression in gastric intestinal metaplasia.

### DETAILED DESCRIPTION OF INVENTION

### I. Overview

The present invention is based, in part, on the discovery that a unique population of primitive epithelial cells give rise to the metaplasia underlying esophageal, gastric, and pancreatic adenocarcinoma and that these cells have a distinct molecular signature.

Specifically, Applicants have demonstrated that during murine embryogenesis, squamous stem cells displace a primitive epithelium in the proximal stomach from the basement membrane to a proliferatively dormant, suprasquamous position. See Wang, X. et al., (2011). Residual embryonic cells are precursors of a Barrett's like metaplasia. Cell 145, 1023-1035, incorporated by reference, herein, in its entirety. However, in mice lacking p63 (a protein that is essential for the self-renewal of stem cells of all stratified epithelial tissues, including mammary and prostate glands as well as all squamous epithelial), these squamous stem cells fail to supplant the primitive epithelium, which then rapidly emerges into a columnar metaplasia with gene expression profiles similar to Barrett's metaplasia but unique to the gastrointestinal tract. Moreover, in adults, a discrete population of these primitive epithelial cells survives embryonic development and resides at the squamocolumnar junction. Upon diphtheria toxin-mediated ablation of squamous epithelial stem cells, these residual embryonic cells begin to invade vacated regions of basement membrane originating a highly proliferative metaplasia. Applicants have further performed histological and gene expression analyses of the metaplasia evident in mouse models of extreme GERD during embryogenesis and in adults to assemble a relative genetic signature of these metaplasias and to define the mechanism of their evolution.

Applicants have also isolated a human Barrett's esophagus progenitor cell. This progenitor cell differentiates into Barrett's esophagus tissue and has a unique mRNA expression profile described below. Together, the clonal population of this Barrett's esophageal progenitor cell allows for the detection and direct therapeutic targeting of the population of cells responsible for the metaplasia by cytotoxic or and/or growth inhibitory agents, thus facilitating the treatment of metaplasia and prevention of its progression to adenocarcinoma. This human Barrett's esophagus progenitor cell can be isolated from human Barrett's metaplasia tissue by dissociating the cells in the tissue and isolating the progenitor cells via FACS using any of proteins described in Table 1, below.

In particular, CDH17 is a novel and specific marker for both Barrett's esophagus stem cells and their differentiated progeny. CDH17 is highly upregulated in BE stem cells. (See Table 3). CDH17 is also different from other existing Barrett's markers, such as Villin and CDX2, which are only detected in differentiated Barrett's and not the stem cells of Barrett's. This makes CDH17 an attractive molecule for diagnosis and targeted therapies for BE stem cells. CDH17 was also detected in gastric intestinal metaplasia and in precursor lesions linked to pancreatic adenocarcinoma. Gastric intestinal metaplasia can also be validated by Villin and Cdx2 staining.

Applicants have also isolated human squamous cell and gastric cardia progenitor cells. Applicants have characterized the mRNA and protein expression of these cells to define these cells and to differentiate their expression profiles from Barrett's esophagus progenitor cells. This allows for the ablation of Barrett's esophagus progenitor cells without reducing the viability of nearby squamous cell or gastric cardia progenitor cells.

Accordingly, the present invention provides methods and compositions for diagnosing, imaging, treating or preventing metaplasia (e.g., esophageal intestinal metaplasia). The present invention also provides methods identifying compounds useful for treating esophageal intestinal metaplasia, gastric intestinal metaplasia, and precursors of pancreatic adenocarcinoma.

### II. Definitions

The term “agent” includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances

As used herein, the term “RNAi agent” refers to an agent, such as a nucleic acid molecule, that mediates gene-silencing by RNA interference, including, without limitation, small interfering siRNAs, small hairpin RNA (shRNA), and microRNA (miRNA).

The term “cell surface receptor ligand”, as used herein, refers to any natural ligand for a cell surface receptor.

The term “antibody” encompasses any antibody (both polyclonal and monoclonal), or fragment thereof, from any animal species. Suitable antibody fragments include, without limitation, single chain antibodies (see e.g., Bird et al. (1988) Science 242:423-426; and Huston et al. (1988) Proc. Natl. Acad. Sci. U.S.A. 85:5879-5883, each of which is herein incorporated by reference in its entirety), domain antibodies (see, e.g., U.S. Pat. Nos. 6,291,158; 6,582,915; 6,593,081; 6,172,197; 6,696,245, each of which is herein incorporated by reference in its entirety), Nanobodies (see, e.g., U.S. Pat. No. 6,765,087, which is herein incorporated by reference in its entirety), and UniBodies (see, e.g., W02007/059782, which is herein incorporated by reference in its entirety

The term “antibody-like molecule”, as used herein, refers to a non-immunoglobulin protein that has been engineered to bind to a desired antigen. Examples of antibody-like molecules include, without limitation, Adnectins (see, e.g., WO 2009/083804, which is herein incorporated by reference in its entirety), Affibodies (see, e.g., U.S. Pat. No. 5,831,012, which is herein incorporated by reference in its entirety), DARPins (see, e.g., U.S. Patent Application Publication No. 2004/0132028, which is herein incorporated by reference in its entirety), Anticalins (see, e.g., U.S. Pat. No. 7,250,297, which is herein incorporated by reference in its entirety), Avimers (see, e.g., U.S. Patent Application Publication Nos. 200610286603, which is herein incorporated by reference in its entirety), and Versabodies (see, e.g., U.S. Patent Application Publication No. 2007/0191272, which is hereby incorporated by reference in its entirety).

The term “cytotoxic moiety”, as used herein, refers to any agent that is detrimental to (e.g., kills) cells.

The term “chemotoxin”, as used herein, refers to any small molecule cytotoxic moiety that is detrimental to (e.g., kills) cells.

The term “biological activity” of a gene, as used herein, refers to a functional activity of the gene or its protein product in a biological system, e.g., enzymatic activity and transcriptional activity.

The term “p63 null mouse”, as used herein, refers to a mouse in which the p63 gene (NCBI Reference Sequence: NM_{—}011641.2) has been deleted or downregulated in one or more tissue (e.g., epithelial tissue).

The term “biocompatible delivery vehicle”, as used herein, refers to any phyioslogically compatible compound that can carry a drug payload, including, without limitation, microcapsules, microparticles, nanoparticles, and liposomes.

The term “imaging moiety”, as used herein, refers to an agent that can be detected and used to image tissue in vivo.

The term “ablated” or “ablation”, as used herein, refers to the functional removal of cells, e.g., the basal cells of the mouse stratified epithelial tissue, using any art-recognized means. In one embodiment, cells are ablated by treatment with a cytotoxic moiety, e.g., using Cre-mediated expression of diphtheria toxin fragment A as described in Ivanova et al. Genesis. 2005; 43:129-35. In other embodiments, cells are chemically or physically ablated, e.g., by endoscopy-assisted ablation, radiofrequency ablation, laser ablation, microwave ablation, cryogenic ablation, thermal ablation, chemical ablation, and the like. In one exemplary embodiment, the ablation energy is radio frequency electrical current applied to conductive needle. The electrical current may be selected to provide pulsed or sinusoidal waveforms, cutting waves, or blended waveforms. In addition, the electrical current may include ablation current followed by current sufficient to cauterize any blood vessels that may be compromised during the ablation process. Alternatively, in some embodiments, ablation probe may take the form of a bipolar probe that carries two or more electrodes, in which case the current flows between the electrodes.

The term “suitable control”, as used herein, refers to a measured mRNA or protein level (e.g. from a tissue sample not subject to treatment by an agent), or a reference value that has previously been established.

The term “pluripotent” as used herein, refers to a stem or progenitor cell that is capable of differentiating into any of the three germ layers endoderm, mesoderm or ectoderm.

The term “multipotent”, as used herein, refers to a stem or progenitor cell that is capable of differentiating into multiple lineages, but not all lineages. Often, multipotent cells can differentiate into most of the cells of a particular lineage, for example, hematopoietic stem cells.

The term “oligopotent”, as used herein, refers to a stem or progenitor cell that can differentiate into two to five cell types, for example, lymphoid or myeloid stem cells.

The term “positive”, as used herein, refers to the expression of an mRNA or protein in a cell, wherein the expression is at least 5 percent of the expression of actin in the cell.

The term “negative”, as used herein, refers to the expression of an mRNA or protein in a cell, wherein the expression is less than 1 percent of the expression of actin in the cell.

### III. Exemplary Embodiments

### A. Molecular Signature of Cells Responsible for the Esophageal Metaplasia.

The present invention is based, in part, on the discovery that a unique population of primitive epithelial cells gives rise to the metaplasia underlying esophageal and gastric adenocarcinoma. Transcriptome analysis of RNA derived by microdissection from this population of cells led to the remarkable discovery that these cells have a distinct molecular signature. In particular, a number of genes were identified as being upregulated in these cells. Moreover, a subset of these genes (set forth below in Table 1, the sequences of which are each specifically incorporated herein by reference to their respective RefSeq Transcript ID numbers) were determined to be useful diagnostically for the identification of these primitive epithelial cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 1
Genes upregulated in Barrett's-like metaplasia

| | | RefSeq |
|---|---|---|
| Gene Symbol | Gene Title | Transcript ID |
| CDH17 | Cadherin 17 | NM_001144663.1 |
| CLRN3 | Clarin 3 | NM_152311.3 |
| TM4SF4 | Transmembrane 4 | NM_004617.3 |
| | L six family | |
| | member 4 | |
| FAM3B | Family with | NM_058186.3 |
| | sequence | |
| | similarity 3, | |
| | member B | |
| NMUR2 | Neuromedin-U | NP_064552.3 |
| | receptor 2 | |
| MUC17 | Mucin-17 | NM_001004430.1 |
| CEACAM7 | Carcinoembryonic | NM_006890.3 |
| | antigen-related | |
| | cell adhesion | |
| | molecule 7 | |
| ANXA13 | Annexin A13 | NM_001003954.1 |
| SLC16A4 | Solute carrier | NM_001201546.1 |
| | family 16, member | |
| | 4 | |
| CD44 | CD44 antigen | NM_000610.3 |
| NPNT | Nephronectin | NM_001184693.1 |
| | | NM_001184692.1 |
| | | NM_001184691.1 |
| | | NM_001184690.1 |
| | | NM_001033047.2 |
| PLBD1 | Phospholipase B | NM_024829.5 |
| | domain containing | |
| | 1 | |
| TFF3 | Trefoil factor 3 | NM_003226.3 |
| REG4 | Regenerating | NM_001159352.1 |
| | islet-derived | |
| | protein 4 | |

Each of the genes shown in Table 1 is also expressed at, at least, 10% of the expression of actin in these cells. These genes were determined to be useful diagnostically for the identification of these cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

These genes, shown in Table 1, are also upregulated in Barrett's esophagus stem cells when compared to their expression in gastric cardia stem cells. These genes were also determined to be useful diagnostically for the identification of these cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal intestinal metaplasia, gastric intestinal metaplasia, intraepithelial pancreatic mucinous neoplasia (IPMN)). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

In certain embodiments, the isolated Barrett's esophagus progenitor cells described herein are negative for the expression of mRNA of any one or more of the genes shown in Table 2, the sequences of which are each specifically incorporated herein by reference to their respective RefSeq Transcript ID numbers.

TABLE 2
Negatively expressing genes

| Gene Symbol | RefSeq |
|---|---|
| SOX2 | NM_003106 |
| TP63 | NM_003722 |
| KRT20 | NM_019010 |
| GKN1 | NM_019517 |
| GKN2 | NM_182536 |
| FABP1 | NM_001443 |
| FABP2 | NM_000134 |
| Krt14 | NM_000526 |
| CXCL17 | NM_198477 |

In certain specific embodiments, the isolated Barrett's esophagus progenitor cells described herein are negative for the expression of Krt20, Sox2 and p63 mRNA. In other specific embodiments, the isolated Barrett's esophagus progenitor cells described herein are negative for the expression of SOX2, p63, KRT20, GKN1, GKN2, FABP1, FABP2, KRT14 and CXCL17.

In certain specific embodiments, the isolated Barrett's esophagus progenitor cells described herein are positive for the expression of CDH17 mRNA. In other specific embodiments, the isolated Barrett's esophagus progenitor cells described herein are negative for the expression of Sox2, p63, Krt20, GKN1/2, FABP1/2, KRT14 and CXCL17 mRNA.

In other embodiments, the isolated Barrett's esophagus progenitor cells described herein are negative for the expression of any one or more of Sox2, p63, Krt20, GKN1/2, FABP1/2, KRT14 or CXCL17 mRNA and positive for the expression of any one or more of CDH17, CLRN3, TM4SF4, FAM3B, NMUR2, MUC17, CEACAM7, ANXA13, SLC16A4, CD44, NPNT, PLBD1, TFF3 and REG4 mRNA. In certain specific embodiments, the isolated Barrett's esophagus progenitor cells described herein are positive for the expression of CDH17 mRNA and negative for the expression of Krt20, Sox2 and p63. In other specific embodiments, the isolated Barrett's esophagus progenitor cells described herein are negative for the expression of Sox2, p63, Krt20, GKN1/2, FABP1/2, KRT14 and CXCL17 mRNA and positive for the expression of CDH17, CLRN3, TM4SF4, FAM3B, NMUR2, MUC17, CEACAM7, ANXA13, SLC16A4, CD44, NPNT, PLBD1, TFF3 and REG4 mRNA.

In certain embodiments, the human isolated clonal population of Barrett's esophagus progenitor cells disclosed herein are cultured with 5 mg/ml insulin, 10 ng/ml EGF, 2×10⁻⁹ M 3,3′,5-triiodo-L-thyronine, 0.4 mg/ml hydrocortisone, 24 mg/ml adenine, 1×10⁻¹⁰ M cholera toxin, 1 μM Jagged 1, 100 ng/ml Noggin, 125 ng/ml R Spondin 1, 2.5 μM Rock inhibitor in DMEM/Ham's F12 3:1 medium with 10% fetal bovine serum when the mRNA expression analysis is performed.

### B. Methods of Treatment

In one aspect, the invention provides methods for treating or preventing metaplasia (e.g., esophageal metaplasia). The methods of the invention generally comprise administering to a subject a therapeutic amount of an agent that decreases the expression and/or biological activity of one or more of the genes set forth in Table 1. In one embodiment, the gene is CDH17.

Any agent that causes a decrease in the expression and/or biological activity of the desired gene(s) is suitable for use in the methods of the invention. Suitable agents include, without limitation, antibodies, antibody-like molecules, aptamers, peptides, antisense oligonucleotides, small molecules or RNAi agents. In some embodiments, the agent decreases the amount of mRNA of the target gene. In other embodiments the agent decreases the expression of the protein product of the targeted gene. In other embodiments, the agent inhibits the biological activity of the protein product of the targeted gene (e.g., enzymatic activity or transcriptional activity). Such agents can be identified, for example, using the screening assays described herein.

In another aspect, the invention provides methods for treating or preventing metaplasia (e.g., esophageal intestinal metaplasia). The methods of the invention generally comprise administering a therapeutic amount of an agent that specifically binds to a cell surface polypeptide encoded by one of the genes set forth in Table 1, wherein said agent is linked to one or more cytotoxic moiety.

Any agent that binds to the desired cell surface polypeptide is suitable for use in the methods of the invention. Suitable agents include, without limitation, antibodies, antibody-like molecules, aptamers, peptides, cell surface receptor ligand, or small molecules. In a preferred embodiment, the agent is an antibody, antibody-like molecule or cell surface receptor ligand. Exemplary antibodies that specifically bind CDH17 can be found in U.S. Patent Publication No. 2010/0092978, incorporated herein by reference in its entirety.

In certain embodiments, cell surface polypeptides are targeted that are highly expressed in the Barrett's esophagus progenitor cell but not in squamous cell progenitor cells that may be located nearby. The squamous cell progenitor cell described above and its mRNA expression profile compared to the profile of the clonal population of Barrett's esophagus progenitor cells.

Any cytotoxic moiety is suitable for use in the methods of the invention, including, without limitation, radioactive isotopes, chemotoxins, or toxin proteins. Suitable radioactive isotopes include, without limitation, iodine¹³¹, indium¹¹¹, yttrium⁹⁰, and lutetium¹⁷⁷. Suitable chemotoxins include, without limitation, anthracyclines (e.g., daunorubicin (formerly daunomycin) and doxorubicin), antibiotics, taxol, cytochalasin B, gramicidin D, ethidium bromide, emetine, mitomycin, etoposide, tenoposide, colchicin, doxorubicin, daunorubicin, dihydroxy anthracin dione, mitoxantrone, mithramycin, actinomycin D, l-dehydrotestosterone, glucocorticoids, procaine, tetracaine, lidocaine, propranolol, antimetabolites (e.g., 30 methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), (e.g., dactinomycin (formerly actinomycin), bleomycin, mithramycin, and anthramycin (AMC)), anti-mitotic agents (e.g., vincristine and vinblastine), duocarmycins, calicheamicins, maytansines and auristatins, and derivatives thereof. Suitable toxin proteins include, without limitation, bacterial toxins (e.g., diphtheria toxin, and plant toxins (e.g., ricin).

In certain embodiments the cytotoxic moiety is linked directly (either covalently or non-covalently) to the agent. In other embodiments the cytotoxic moiety is incorporated into a biocompatible delivery vehicle that is in turn linked directly (either covalently or non-covalently) to the agent. Biocompatible delivery vehicles are well known in the art and include, without limitation, microcapsules, microparticles, nanoparticles, liposomes and the like.

Applicants have discovered that it is a primitive cell population residing at the squamocolumnar junction that is responsible for esophageal metaplasia. Accordingly, ablation of this cell population in normal, healthy individuals would protect those individuals from esophageal metaplasia and, in turn, from esophageal adenocarcinoma. Thus, the present invention provides for both prophylactic and therapeutic methods of treatment. In some embodiments, the patient to be treated has been diagnosed as having metaplasia. In other embodiments, the patient to be treated does not have metaplasia.

According to the methods of the invention, the agent can be administered via any means appropriate to effect treatment. In some embodiments, the agent is administered parenterally. In other embodiments, the agent is administered orally. In a preferred embodiment, the agent is administered endoscopically to the esophageal squamocolumnar junction or to a site of esophageal metaplasia. Any endoscopic device or procedure capable of delivering an agent is suitable for use in the methods of the invention.

An agent of the invention typically is administered to the subject in a pharmaceutical composition. The pharmaceutical composition typically includes the agent formulated together with a pharmaceutically acceptable carrier. Pharmaceutical compositions can be administered in combination therapy, i.e., combined with other agents. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for oral, and parenteral administration (e.g., by injection or infusion).

In some embodiments, the expression of genes required for activation, division or growth of the stem cell can reduced or otherwise inhibited using a nucleic acid therapeutic. In preferred embodiments, the nucleic acid therapeutic is selectively cytotoxic or cytotoxic to the stem cell relative to other normal tissue in the alimentary canal, particularly adjacent tissues. In the case of the BE stem cell, preferable nucleic acid therapeutics are selectively cytotoxic or cytotoxic to the BE cell as relative to normal esophageal squamous epithelium and/or esophageal squamous stem cells and/or stomach cardia stem cells.

Exemplary nucleic acid therapeutics include, but are not limited to, antisense oligonucleotides, decoys, siRNAs, miRNAs, shRNAs and ribozymes. These agents can be delivered through a variety of routes of administration, but a preferred route is through local delivery, such as by local injection or endoscopic delivery. Moreover, the nucleic acid therapeutic can be modified with one or more moieties which promote uptake of the polynucleotide by the targeted stem cell. For instance, the modification can be a peptide or a peptidomimetic that enhances cell permeation, or a lipophilic moiety which enhances entrance into a cell. Exemplary lipophilic moieties include those chosen from the group consisting of a lipid, cholesterol, oleyl, retinyl, cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

RNA Interference Nucleic Acids

In particular embodiments, nucleic acid therapeutic is an RNA interference (RNAi) molecule. RNA interference methods using RNAi molecules may be used to disrupt the expression of a gene of interest, such as gene overexpressed by the targeted stem cell. Exemplary genes to be targeted in the case of BE stem cells are provided in Table 1. In one embodiment, the gene is CDH17. Exemplary RNAi molecules for disruption of expression of CDH17 can be found in U.S. Patent Publication No. 2010/0092978, incorporated herein by reference in its entirety. Small interfering RNA (siRNA) are RNA duplexes normally 21-30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). Thus, the invention includes the use of RNAi molecules comprising any of these different types of double-stranded molecules. In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded polynucleotides comprising two separate strands, i.e. a sense strand and an antisense strand, e.g., small interfering RNA (siRNA); polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

RNA interference (RNAi) may be used to specifically inhibit expression of target genes in the stem cell. Double-stranded RNA-mediated suppression of gene and nucleic acid expression may be accomplished according to the invention by introducing dsRNA, siRNA or shRNA into cells or organisms. SiRNA may be double-stranded RNA, or a hybrid molecule comprising both RNA and DNA, e.g., one RNA strand and one DNA strand. It has been demonstrated that the direct introduction of siRNAs to a cell can trigger RNAi in mammalian cells (Elshabir, S. M., et al. Nature 411:494-498 (2001)). Furthermore, suppression in mammalian cells occurred at the RNA level and was specific for the targeted genes, with a strong correlation between RNA and protein suppression (Caplen, N. et al., Proc. Natl. Acad. Sci. USA 98:9746-9747 (2001)).

RNAi molecules targeting specific genes can be readily prepared according to procedures known in the art. Structural characteristics of effective siRNA molecules have been identified. Elshabir, S. M. et al. (2001) Nature 411:494-498 and Elshabir, S. M. et al. (2001), EMBO 20:6877-6888. Accordingly, one of skill in the art would understand that a wide variety of different siRNA molecules may be used to target a specific gene or transcript. In certain embodiments, siRNA molecules according to the invention are double-stranded and 16-30 or 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs.

Generally, siRNA molecules are completely complementary to the target mRNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al. EMBO J. 20:6877-6888 (2001)). In addition, potential target sites may be compared to an appropriate genome database, such as BLASTN 2.0.5, available on the NCBI server at www.ncbi.nlm, and potential target sequences with significant homology to other coding sequences eliminated.

Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing works in a variety of normal and cancer cell lines, and in mammalian cells, including mouse and human cells. Paddison, P. et al., Genes Dev. 16(8):948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., Proc. Natl. Acad. Sci. USA 99(3):1443-1448 (2002).

ShRNAs contain a stem loop structure. In certain embodiments, they may contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) Genes & Dev. 16(8):948-58).

Micro RNAs

In other embodiments, the nucleic acid therapeutic is a Micro RNA (miRNA), Micro RNA mimic or an antagonist. Micro RNAs (miRNAs) are a highly conserved class of small RNA molecules that are transcribed from DNA in the genomes of plants and animals, but are not translated into protein. Processed miRNAs are single stranded 17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111; and also at http://microrna.sanger.ac.uk/sequences/. In certain preferred embodiments, the miRNA, miRNA mimic or antagonist is selectively cytotoxic or cytotoxic to BE cell as relative to normal esophageal squamous epithelium and/or esophageal squamous stem cells and/or gastric cardia stem cells.

Antisense Oligonucleotides

In one embodiment, the nucleic acid therapeutic is an antisense oligonucleotide directed to a target gene overexpressed in the stem cell, i.e., the BE stem cell, or for which inhibition of expression is selectively cytotoxic or cytotoxic to the BE cell as relative to normal esophageal squamous epithelium and/or esophageal squamous stem cells and/or stomach cardia stem cells. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence. In the case of antisense RNA, they prevent translation of complementary RNA strands by binding to it. Antisense DNA can be used to target a specific, complementary (coding or non-coding) RNA. If binding takes places this DNA/RNA hybrid can be degraded by the enzyme RNase H. In particular embodiment, antisense oligonucleotides contain from about 10 to about 50 nucleotides, more preferably about 15 to about 30 nucleotides. The term also encompasses antisense oligonucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tₘ, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

Ribozymes

According to another embodiment of the invention, the nucleic acid therapeutic is a ribozyme. Ribozymes are RNA-protein complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20) and can cleave an inactive a target mRNA. For example, a large number of ribozymes accelerate phosphodiester transfer reactions with a high degree of specificity, often cleaving only one of several phosphodiesters in an oligonucleotide substrate (Cech et al., Cell. 1981 December; 27(3 Pt 2):487-96; Michel and Westhof, J Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Desirable characteristics of enzymatic nucleic acid molecules used according to the invention are that they have a specific substrate binding site which is complementary to one or more of the target RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Cell Penetrating Moieties Attached to the Nucleic Acid Therapeutics

A variety of agents can be associated with the nucleic acid therapeutic, preferably through a reversible covalent linker, in order to enhance the uptake of the therapeutic by cells, particularly the targeted stem cell. These cell penetrating (CP) moieties may be so attached directly or indirectly via a linker. Functionally, the CP moieties may be designed to achieve one or more improved outcomes. As used herein the term “CP moiety” is a compound or molecule or construct which is attached, linked or associated with the nucleic acid therapeutic.

In one embodiment the CP moieties comprise molecules which promote endocytosis of the nucleic acid therapeutic. As such the CP moiety acts as a “membrane intercalator.” For example, the membrane intercalators may comprise C₁₀-C₁₈ moieties which may be attached to the 3′ end of antisense strand. These moieties may facilitate or result in the nucleic acid therapeutic becoming embedded in the lipid bilayer of a cell. Upon “flipping” of the lipids, the nucleic acid therapeutic would then enter the cell. In these constructs, the linker between the CP moiety and the nucleic acid therapeutic can be selected such that it is sensitive to the physicochemical environment of the cell and/or to be susceptible to or resistant to enzymes present. The end result being the liberation of the nucleic acid therapeutic, with or without a portion of the optional linker. The present invention also contemplates nucleic acid therapeutics that bind to receptors which are internalized.

Furthermore, the nucleic acid therapeutics of the invention itself can have one or more CP moieties which facilitates the active or passive transport, localization, or compartmentalization of the nucleic acid therapeutic.

Conjugates as CP Moieties

CP moieties, while attached directly to the nucleic acid therapeutic or to the nucleic acid therapeutic via an optional linker may comprise conjugate groups attached to one or more of the nucleic acid therapeutic termini at selected nucleobase positions, sugar positions or to one of the terminal internucleoside linkages.

There are numerous methods for preparing conjugates of nucleic acid therapeutics. Generally, a nucleic acid therapeutic is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic. For example, an electrophilic group can be a carbonyl-containing functionality and a nucleophilic group can be an amine or thiol. Methods for conjugation of nucleic acids and related compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In some embodiments, conjugate moieties can be attached to the terminus of a nucleic acid therapeutic such as a 5′ or 3′ terminal residue of either strand. Conjugate moieties can also be attached to internal residues of the oligomeric compounds. For nucleic acid therapeutics, conjugate moieties can be attached to one or both strands. In some embodiments, a double-stranded nucleic acid therapeutic contains a conjugate moiety attached to each end of the sense strand. In other embodiments, a double-stranded nucleic acid therapeutic contains a conjugate moiety attached to both ends of the antisense strand.

In some embodiments, conjugate moieties can be attached to heterocyclic base moieties (e.g., purines and pyrimidines), monomeric subunits (e.g., sugar moieties), or monomeric subunit linkages (e.g., phosphodiester linkages) of nucleic acid molecules. Conjugation to purines or derivatives thereof can occur at any position including, endocyclic and exocyclic atoms. In some embodiments, the 2-, 6-, 7-, or 8-positions of a purine base are attached to a conjugate moiety. Conjugation to pyrimidines or derivatives thereof can also occur at any position. In some embodiments, the 2-, 5-, and 6-positions of a pyrimidine base can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms.

Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

These CP moieties act to enhance the properties of the nucleic acid therapeutic or may be used to track the nucleic acid therapeutic or its metabolites and/or effect the trafficking of the construct. Properties that are typically enhanced include without limitation activity, cellular distribution and cellular uptake. In one embodiment, the nucleic acid therapeutics are prepared by covalently attaching the CP moieties to chemically functional groups available on the nucleic acid therapeutic or linker such as hydroxyl or amino functional groups. Conjugates which may be used as terminal moieties include intercalators, reporter molecules, polyamines, polyamides, polyethylene glycols, polyethers, and groups that enhance the pharmacodynamic and/or pharmacokinetic properties of the nucleic acid therapeutic.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve properties including but not limited to construct uptake, construct resistance to degradation, and/or strengthen sequence-specific hybridization with RNA.

Conjugate groups also include but are not limited to lipid moieties such as a cholesterol moiety, cholic acid, a thioether, an aliphatic chain, a phospholipid, a polyamine or a polyethylene glycol chain or adamantane acetic acid, a palmityl moiety or an octadecylamine or hexylamino-carbonyl-oxycholesterol moiety.

The nucleic acid therapeutics of the invention may also be conjugated to active drug substances. Representative U.S. patents that teach the preparation of such conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The present invention provides, inter alia, nucleic acid therapeutics and compositions containing the same wherein the CP moiety comprises one or more conjugate moieties. The CP moieties (e.g., conjugates) of the present invention can be covalently attached, optionally through one or more linkers, to one or more nucleic acid therapeutics. The resulting constructs can have modified or enhanced pharmacokinetic, pharmacodynamic, and other properties compared with non-conjugated constructs. A conjugate moiety that can modify or enhance the pharmacokinetic properties of a nucleic acid therapeutic can improve cellular distribution, bioavailability, metabolism, excretion, permeability, and/or cellular uptake of the nucleic acid therapeutic. A conjugate moiety that can modify or enhance pharmacodynamic properties of a nucleic acid therapeutic can improve activity, resistance to degradation, sequence-specific hybridization, uptake, and the like.

Representative conjugate moieties can include lipophilic molecules (aromatic and non-aromatic) including steroid molecules; proteins (e.g., antibodies, enzymes, serum proteins); peptides; vitamins (water-soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. Oligonucleotide conjugates and their syntheses are also reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense & Nucleic Acid Drug Development, 2002, 12, 103, each of which is incorporated herein by reference in its entirety.

Lipophilic conjugate moieties can be used, for example, to counter the hydrophilic nature of a nucleic acid therapeutic and enhance cellular penetration. Lipophilic moieties include, for example, steroids and related compounds such as cholesterol (U.S. Pat. No. 4,958,013 and Letsinger et al., Proc. Natl. Acad. Sci. USA, 1989, 86, 6553), thiocholesterol (Oberhauser et al., Nuc. Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosanoic acids and the like.

In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like.

Other suitable lipophilic conjugate moieties include menthols, trityls (e.g., dimethoxytrityl (DMT)), phenoxazines, lipoic acid, phospholipids, ethers, thioethers (e.g., hexyl-5-tritylthiol), derivatives thereof and the like. nucleic acid therapeutics containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump et al., Bioconjugate Chem. 9: 341, 1998; Firestone, Bioconjugate Chem. 5: 105, 1994; Mishra et al., Biochim. Biophys. Acta 1264: 229, 1995). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

Conjugate moieties can also include vitamins. Vitamins are known to be transported into cells by numerous cellular transport systems. Typically, vitamins can be classified as water soluble or lipid soluble. Water soluble vitamins include thiamine, riboflavin, nicotinic acid or niacin, the vitamin B₆ pyridoxal group, pantothenic acid, biotin, folic acid, the B₁₂ cobamide coenzymes, inositol, choline and ascorbic acid. Lipid soluble vitamins include the vitamin A family, vitamin D, the vitamin E tocopherol family and vitamin K (and phytols).

In some embodiments, the conjugate moiety includes folic acid (folate) and/or one or more of its various forms, such as dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates.

Vitamin conjugate moieties include, for example, vitamin A (retinol) and/or related compounds. The vitamin A family (retinoids), including retinoic acid and retinol, are typically absorbed and transported to target tissues through their interaction with specific proteins such as cytosol retinol-binding protein type II (CRBP-II), retinol binding protein (RBP), and cellular retinol-binding protein (CRBP). The vitamin A family of compounds can be attached to a nucleic acid therapeutic via acid or alcohol functionalities found in the various family members. For example, conjugation of an N-hydroxy succinimide ester of an acid moiety of retinoic acid to an amine function on a linker pendant to a nucleic acid therapeutic can result in linkage of vitamin A compound to the nucleic acid therapeutic via an amide bond. Also, retinol can be converted to its phosphoramidite, which is useful for 5′ conjugation.

alpha-Tocopherol (vitamin E) and the other tocopherols (beta through zeta) can be conjugated to nucleic acid therapeutics to enhance uptake because of their lipophilic character. Also, vitamin D, and its ergosterol precursors, can be conjugated to nucleic acid therapeutics through their hydroxyl groups by first activating the hydroxyl groups to, for example, hemisuccinate esters. Conjugation can then be effected directly to the nucleic acid therapeutic or to an amino linker pendant from the nucleic acid therapeutic. Other vitamins that can be conjugated to nucleic acid therapeutics in a similar manner on include thiamine, riboflavin, pyridoxine, pyridoxamine, pyridoxal, deoxypyridoxine. Lipid soluble vitamin K's and related quinone-containing compounds can be conjugated via carbonyl groups on the quinone ring. The phytol moiety of vitamin K can also serve to enhance binding of the oligomeric compounds to cells.

Pyridoxal (vitamin B₆) has specific B₆-binding proteins. Other pyridoxal family members include pyridoxine, pyridoxamine, pyridoxal phosphate, and pyridoxic acid. Pyridoxic acid, niacin, pantothenic acid, biotin, folic acid and ascorbic acid can be conjugated to nucleic acid therapeutics, for example, using N-hydroxysuccinimide esters that are reactive with amino linkers located on the nucleic acid therapeutic, as described above for retinoic acid.

Conjugate moieties can also include polymers. Polymers can provide added bulk and various functional groups to affect permeation, cellular transport, and localization of the conjugated nucleic acid therapeutic. For example, increased hydrodynamic radius caused by conjugation of a nucleic acid therapeutic with a polymer can help prevent entry into the nucleus and encourage localization in the cytoplasm. In some embodiments, the polymer does not substantially reduce cellular uptake or interfere with hybridization to a complementary strand or other target. In further embodiments, the conjugate polymer moiety has, for example, a molecular weight of less than about 40, less than about 30, or less than about 20 kDa. Additionally, polymer conjugate moieties can be water-soluble and optionally further comprise other conjugate moieties such as peptides, carbohydrates, drugs, reporter groups, or further conjugate moieties.

In some embodiments, polymer conjugates include polyethylene glycol (PEG) and copolymers and derivatives thereof. Conjugation to PEG has been shown to increase nuclease stability of nucleic acid based compounds. PEG conjugate moieties can be of any molecular weight including for example, about 100, about 500, about 1000, about 2000, about 5000, about 10,000 and higher. In some embodiments, the PEG conjugate moieties contains at least 4, at least 5, at least 6, at least 7, at least 8, at least 9, at least 10, at least 15, at least 20, or at least 25 ethylene glycol residues. In further embodiments, the PEG conjugate moiety contains from about 4 to about 10, about 4 to about 8, about 5 to about 7, or about 6 ethylene glycol residues. The PEG conjugate moiety can also be modified such that a terminal hydroxyl is replaced by alkoxy, carboxy, acyl, amido, or other functionality. Other conjugate moieties, such as reporter groups including, for example, biotin or fluorescein can also be attached to a PEG conjugate moiety. Copolymers of PEG are also suitable as conjugate moieties. Preparation and biological activity of polyethylene glycol conjugates of oligonucleotides are described, for example, in Bonora et al., Nucleosides Nucleotides 18: 1723, 1999; Bonora et al., Farmaco 53: 634, 1998; Efimov, Bioorg. Khim. 19: 800, 1993; and Jaschke et al., Nucleic Acids Res. 22: 4810, 1994. Further example PEG conjugate moieties and preparation of corresponding conjugated oligomeric compounds is described in, for example, U.S. Pat. Nos. 4,904,582 and 5,672,662, each of which is incorporated by reference herein in its entirety. Nucleic acid compounds conjugated to one or more PEG moieties are available commercially.

Other polymers suitable as conjugate moieties include polyamines, polypeptides, polymethacrylates (e.g., hydroxylpropyl methacrylate (HPMA)), poly(L-lactide), poly(DL lactide-co-glycolide (PGLA), polyacrylic acids, polyethylenimines (PEI), polyalkylacrylic acids, polyurethanes, polyacrylamides, N-alkylacrylamides, polyspermine (PSP), polyethers, cyclodextrins, derivatives thereof and co-polymers thereof. Many polymers, such as PEG and polyamines have receptors present in certain cells, thereby facilitating cellular uptake. Polyamines and other amine-containing polymers can exist in protonated form at physiological pH, effectively countering an anionic backbone of some oligomeric compounds, effectively enhancing cellular permeation. Some example polyamines include polypeptides (e.g., polylysine, polyomithine, polyhistadine, polyarginine, and copolymers thereof), triethylenetetramine, spermine, polyspermine, spermidine, synnorspermidine, C-branched spermidine, and derivatives thereof. Other amine-containing moieties can also serve as suitable conjugate moieties due to, for example, the formation of cationic species at physiological conditions. Example amine-containing moieties include 3-aminopropyl, 3-(N,N-dimethylamino)propyl, 2-(2-(N,N-dimethylamino)ethoxy)ethyl, 2-N-(2-aminoethyl)-N-methylaminooxy)ethyl, 2-(1-imidazolyl)ethyl, and the like.

Conjugate moieties can also include peptides. Suitable peptides can have from 2 to about 30, 2 to about 20, 2 to about 15, or 2 to about 10 amino acid residues. Amino acid residues can be naturally or non-naturally occurring, including both D and L isomers.

In some embodiments, peptide conjugate moieties are pH sensitive peptides such as fusogenic peptides. Fusogenic peptides can facilitate endosomal release of agents such as nucleic acid therapeutics to the cytoplasm. It is believed that fusogenic peptides change conformation in acidic pH, effectively destabilizing the endosomal membrane thereby enhancing cytoplasmic delivery of endosomal contents. Example fusogenic peptides include peptides derived from polymyxin B, influenza HA2, GALA, KALA, EALA, melittin-derived peptide, α-helical peptide or Alzheimer β-amyloid peptide, and the like. Preparation and biological activity of oligonucleotides conjugated to fusogenic peptides are described in, for example, Bongartz et al., Nucleic Acids Res. 22: 4681, 1994, and U.S. Pat. Nos. 6,559,279 and 6,344,436.

Other peptides that can serve as conjugate moieties include delivery peptides which have the ability to transport relatively large, polar molecules (including peptides, oligonucleotides, and proteins) across cell membranes. Example delivery peptides include Tat peptide from HIV Tat protein and Ant peptide from Drosophila antenna protein. Conjugation of Tat and Ant with oligonucleotides is described in, for example, Astriab-Fisher et al., Biochem. Pharmacol. 60: 83, 2000.

Conjugated delivery peptides can help control localization of nucleic acid therapeutics and constructs to specific regions of a cell, including, for example, the cytoplasm, nucleus, nucleolus, and endoplasmic reticulum (ER). Nuclear localization can be effected by conjugation of a nuclear localization signal (NLS). In contrast, cytoplasmic localization can be facilitated by conjugation of a nuclear export signal (NES). Methods for conjugating peptides to oligomeric compounds such as oligonucleotides is described in, for example, U.S. Pat. No. 6,559,279, which is incorporated herein by reference in its entirety.

Many drugs, receptor ligands, toxins, reporter molecules, and other small molecules can serve as conjugate moieties. Small molecule conjugate moieties often have specific interactions with certain receptors or other biomolecules, thereby allowing targeting of conjugated nucleic acid therapeutics to specific cells or tissues.

Other conjugate moieties can include proteins, subunits, or fragments thereof. Proteins include, for example, enzymes, reporter enzymes, antibodies, receptors, and the like. In some embodiments, protein conjugate moieties can be antibodies or fragments. Antibodies can be designed to bind to desired targets such as tumor and other disease-related antigens. In further embodiments, protein conjugate moieties can be serum proteins. In yet further embodiments, nucleic acid therapeutics can be conjugated to RNAi-related proteins, RNAi-related protein complexes, subunits, and fragments thereof. For example, oligomeric compounds can be conjugated to Dicer or RISC or fragments thereof. RISC is a ribonucleoprotein complex that contains an oligonucleotide component and proteins of the Argonaute family of proteins, among others. Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNase, Dicer.

Other conjugate moieties can include, for example, oligosaccharides and carbohydrate clusters; a glycotripeptide that binds to Gal/GalNAc receptors on hepatocytes, lysine-based galactose clusters; and cholane-based galactose clusters (e.g., carbohydrate recognition motif for asialoglycoprotein receptor). Further suitable conjugates can include oligosaccharides that can bind to carbohydrate recognition domains (CRD) found on the asialoglycoprotein-receptor (ASGP-R).

A wide variety of linker groups are known in the art that can be useful in the attachment of CP moieties to nucleic acid therapeutics. A review of many of the useful linker groups can be found in, for example, Antisense Research and Applications, S. T. Crooke and B. Lebleu, Eds., CRC Press, Boca Raton, Fla., 1993, p. 303-350. Any of the reported groups can be used as a single linker or in combination with one or more further linkers.

Linkers and their use in preparation of conjugates of oligonucleotides are provided throughout the art. For example, see U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510,475; 5,512,667; 5,574,142; 5,684,142; 5,770,716; 6,096,875; 6,335,432; and 6,335,437.

In one embodiment, the linker may comprise a nucleic acid hairpin which links the 5′ end of one strand

The term “linking moiety,” or “linker” as used herein is generally a bi-functional group, molecule or compound. It may covalently or non-covalently bind the nucleic acid therapeutic to the CP moiety. The covalent binding may be at both or only one end of the linker. Whether the nature of binding to the nucleic acid therapeutic and CP moiety is either covalent or noncovalent, the linker itself may be labile. As used herein, the term “labile” as it applies to linkers means that the linker is either temporally or spatially stable for only a definite period or under certain environmental conditions. For example, a labile linker may lose integrity at a certain, time, temperature, pH, pressure, or under a certain magnetic field or electric field. The result of lost integrity may be the severance of the connection between the nucleic acid therapeutic and one or more CP moieties.

Suitable linking moieties or linkers include, but are not limited to, divalent group such as alkylene, cycloalkylene, arylene, heterocyclyl, heteroarylene, and the other variables are as described herein.

### C. Imaging Methods

In another aspect, the invention provides methods for imaging metaplasia (e.g., esophageal intestinal metaplasia). The methods of the invention generally comprise administering to a subject an effective amount of an agent that specifically binds to a cell surface polypeptide encoded by one of the genes set forth in Table 1, and visualizing the agent. In one embodiment, the gene is CDH17. In a preferred embodiment, cell surface proteins are used that are differentially expressed in Barrett's esophagus progenitor cells and squamous cell progenitor cells and/or gastric cardia progenitor cells.

Any agent that binds to the desired cell surface polypeptide is suitable for use in the methods of the invention. Suitable agents include, without limitation, antibodies, aptamers, peptides, cell surface receptor ligands, or small molecules. In a preferred embodiment, the agent is an antibody, antibody-like molecule or cell surface receptor ligand. Exemplary antibodies that specifically bind CDH17 can be found in U.S. Patent Publication No. 2010/0092978, incorporated herein by reference in its entirety.

In certain embodiments, the invention provides an imaging agent useful for optical coherence tomography (OCT) imaging, which agent includes a moiety (such as an antibody) that specifically binds to a BE stem cell and/or BE metaplasia cell and an OCT detection/contrast agent for enhancing detection by OCT of tissue to which the detection binds. The phrase “enhancing detection by OCT” means that an image produced by OCT with the enhancement shows a greater difference in optical properties between parts of the image, than an otherwise identical image produced without the enhancement. An “OCT contrast agent” means any substance that changes the optical properties of tissue containing the substance. Optical properties that may be changed include absorbance, reflectance, fluorescence, birefringence and optical scattering. OCT contrast agents which can be associated with a detection agent include near-infrared dyes, polypyrrole nanoparticles (see, for example, Au et al. Advanced Materials (2011) 23:5792, incorporated by reference herein in its entirety), engineered microsphere contrast agents (see for example Lee et al. (2003) Optics Letters 28:1546, incorporated by reference herein in its entirety).

In certain embodiments, the imaging agent is a Positron Emission Tomography (PET) detection/contrast agent or a magnetic resonance imaging (MRI) detection/contrast agent. An MRI detection/contrast agent can comprise a paramagnetic contrast agent (such as a gadolinium compound), a super paramagnetic contrast agent (such as iron oxide nanoparticles), a diamagnetic agent (such as barium sulfate), and combinations thereof. Metal ions preferred for MRI include those with atomic numbers 21-29, 39-47, or 57-83, and, more preferably, a paramagnetic form of a metal ion with atomic numbers 21-29, 42, 44, or 57-83. Particularly preferred paramagnetic metal ions are selected from the group consisting of Gd(III), Fe(III), Mn(II and III), Cr(III), Cu(II), Dy(III), Tb(III and IV), Ho(III), Er(III), Pr(III) and Eu(II and III). Gd(III) is particularly useful. Note that as used herein, the term “Gd” is meant to convey the ionic form of the metal gadolinium; such an ionic form can be written as GD(III), GD3+, etc. with no difference in ionic form contemplated.

MRI detection/contrast agents and PET imaging agents can further include ⁶⁴Cu diacetyl-bis(N-4-methylthiosemicarbazone) (⁶⁴Cu-ATSM). It is also referred to as ATSM or Copper 64. PET imaging agents can also include ¹⁸F-fluorodeoxyglucose (FDG) is a radioactive sugar molecule, that, when used with PET imaging, produces images that show the metabolic activity of tissues. In FDG-PET scanning, the high consumption of the sugar by tumor cells, as compared to the lower consumption by normal surrounding tissues, can identify these cells as cancer cells. FDG is also used to study tumor response to treatment. PET imaging agents can also include ¹⁸F-fluoride. This agent can assess changes both in normal bone as well as bone tumors. In certain embodiments, it can be used to measure response to treatment. PET imaging agents can also include 3′-deoxy-3′-[¹⁸F]fluorothymidine (FLT). This agent is a radiolabeled imaging agent that is able to detect growth in a primary tumor. PET imaging agents can also include ¹⁸F-fluoromisonidazole (FMISO) is an imaging agent used with PET imaging that can identify hypoxia in tissues. Tumors with low oxygen have been shown to be resistant to radiation and chemotherapy. PET imaging agents can also include Gallium. Gallium attaches to areas of inflammation, such as infection. It also attaches to areas of rapid cell division, such as cancer cells. It can take gallium a few days to accumulate in the affected tissue, so the scan may be done 2-3 days after the gallium is administered. PET imaging agents can also include technetium-99m. This agent is used to radiolabel many different common radiopharmaceuticals. It is used most often in bone and heart scans. PET imaging agents can also include Thallium. Thallium is a radioactive tracer typically used to examine heart blood flow. The thallium scan is often combined with an exercise test to determine how well the heart functions under stress. A thallium scan may also be used to measure tumor response.

In certain embodiments, these MRI and PET detection/contrast and imaging agents are linked (covalently or non-covalently) with an imaging agent that specifically binds to a BE stem cell specific protein. These proteins include, but are not limited to CDH17, CLRN3, TM4SF4, FAM3B, NMUR2, MUC17, CEACAM7, ANXA13, SLC16A4, CD44, NPNT, PLBD1, TFF3 and REG4. In a specific embodiment, the protein is CDH17. In some embodiments, the agent is linked (covalently or non-covalently) to an imaging moiety to facilitate detection of the agent. Any imaging moiety is suitable for use in the methods of the invention, including, without limitation, positron-emitters, nuclear magnetic resonance spin probes, an optically visible dye, or an optically visible particle. Suitable positron-emitters include, without limitation, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. Suitable nuclear magnetic resonance spin probes include, without limitation, iron chelates and radioactive chelates of gadolinium or manganese.

Exemplary detection/contrast enhancing agents for MRI include, but are not limited to: ferro, antiferro, ferrimagnetic or superparamagnetic material such as iron (Fe), iron oxide γ-Fe₂O₃ or Fe₃O₄ or ferrite with spinel structure MFe₂O₄ (M=Mn, Co, Ni, Cu, Zn, Cd) or ferrite with garnet structure M₃Fe₅O₁₂ (M=Y, La, Ce, Pr, Nd, Sm, Eu, Gd, Th, Dy, Ho, Er, Tm, Yb, Lu) or ferrite with a magnetoplumbite structure MFe₁₂O₁₉ (M=Ca, Sr, Ba, Zn) or other hexagonal ferrite structures such as e.g. Ba₂M₂Fe₁₂O₂₂ (M=Mn, Fe, Co, Ni, Zn, Mg); in all cases, the core can be doped with additional 0.01 to 5.00 mol % of Mn, Co, Ni, Cu, Zn or F.

In other embodiments the detection/contrast agent can be paramagnetic ions (e.g. lanthanide, manganese, iron, copper)-based contrast-enhancing units, e.g. gadolinium chelates such as Gd(DTPA), Gd(BMA-DTPA), Gd(DOTA), Gd(DO3A); oligomeric structures; macromolecular structures such as albumin Gd(DTPA)20-35, dextran Gd(DTPA), Gd(DTPA)-24-cascade polymer, polylysine-Gd(DTPA), MPEG polylysine-Gd(DTPA); dendrimeric structures of lanthanide-based contrast-enhancing units; manganese-based contrast-enhancing units such as Mn(DPDP), Mn(EDTA-MEA), poly-Mn(EED-EEA), and polymeric structures; liposomes as carriers of paramagnetic ions, e.g. liposomal Gd(DTPA); non-proton imaging agents.

Exemplary optical detection/contrast agents include, but are not limited to luminescent materials such as nanophosphors (e.g. rare-earth doped YPO₄ or LaPO₄) or semiconducting nanocrystals (referred to as quantum dots; e.g. CdS, CdSe, ZnS/CdSe, ZnS/CdS); carbocyanine dyes; tetrapyrrole-based dyes (porphyrins, chlorins, phthalocyanines and related structures); delta aminolevulinic, acid; fluorescent lanthanide chelates; fluorescein or 5-aminofluorescein or fluorescein isothiocyanate (FITC) or other fluorescein-related fluorophors such as Oregon Green, naphthofluorescein.

Exemplary ultrasounds detection/contrast agents include but are not limited to shell (e.g. protein, lipid, surfactant or polymer) encapsulated gas (e.g. air, perfluoropropane, dodecafluorocarbon, sulphur hexafluoride, perfluorocarbon) bubbles (such as Optison from Amersham, Levovist from Schering); shell (e.g. protein, lipid, surfactant or polymer) encapsulated droplets; nanoparticles (e.g. platinum, gold, tantalum).

Exemplary x-ray detection/contrast agents include but are not limited to iodinated contrast-enhancing units such as e.g. ionic and non-ionic derivatives of 2,4,6-tri-iodobenzene; barium sulfate-based contrast-enhancing units; metal on chelates such as e.g. gadolinium-based compounds; boron clusters with a high proportion of iodine; polymers like iodinated polysaccharides, polymeric triiodobenzenes; particles from iodinated compounds displaying low water solubility; liposomes containing iodinated compounds; iodinated lipids such as triglycerides, fatty acids.

Exemplary PET detection/contrast agents include but are not limited to ¹¹C, ¹³N, ¹⁵O, ^{66/8}Ga, ⁶⁰Cu, ⁵²Fe, ⁵⁵Co, ⁶¹Cu, ⁶²Cu, ⁶⁴Cu, ⁶²Zn, ⁶³Zn, ⁷⁰As, ⁷¹As, ⁷⁴AS, ⁷⁵Br, ⁷⁶Br, ⁸²Rb, ⁸⁶Y, ⁸⁹Zr, ¹¹⁰In, ¹²⁰I, ¹²⁴I, ¹²²Xe and ¹⁸F-based tracers.

Exemplary SPECT detection/contrast agents include but are not limited to contrast-enhancing units based on radionucleotides such as e.g. ⁹⁹mTc, ¹²³I, ¹³¹I, ⁶⁷Cu, ¹¹¹In, ²⁰¹Tl.

In certain embodiments, ablation techniques are used in conjunction with imaging methods disclosed herein. For example, the expression markers described herein may improve the ability to image or otherwise visualize metaplastic cells and facilitate their ablation. The types of ablation technique that techniques that be used in conjunction with imaging or other visualization of markers described herein include radiofrequency, laser, microwave, cryogenic, thermal, chemical, and the like. The ablation probe may conform to the ablation energy source. For example, an endoscope with fiber optics can be used to view the operation field, and to help select the areas for ablation based on the detection of one or more markers described here.

### D. Diagnostic Methods

In another aspect, the invention provides methods for diagnosing, or predicting the future development of metaplasia (e.g., esophageal intestinal metaplasia). The methods of the invention generally comprise measuring the expression level of one or more of the genes set forth in Table 1 in an epithelial tissue sample from a subject, wherein an increase in the expression level relative to a suitable control indicates that the subject has, or has a future risk of developing, metaplasia. In one embodiment, the gene is CDH17. In a preferred embodiment, cell surface proteins are used that are differentially expressed in Barrett's esophagus progenitor cells and squamous cell progenitor cells.

Any means for measuring the expression level of a gene is suitable for use in the methods of the invention. Exemplary, art recognized, methods include, without limitation, gene expression profiling using gene chips to detect mRNA levels or antibody-based binding assays (e.g. ELISA) to detect the protein-product of a gene. Exemplary antibodies that specifically bind CDH17 can be found in U.S. Patent Publication No. 2010/0092978, incorporated herein by reference in its entirety.

The epithelial tissue sample can be obtained by any means, including biopsy or by scraping or swabbing an area or by using a needle to aspirate. Methods for collecting various body samples are well known in the art, including, without limitation, endoscopic biopsy. Tissue samples may be fresh, frozen, or fixed according to methods known to one of skill in the art.

The diagnostic methods of the invention are generally performed in vitro. However, in certain embodiments, the tissue sample is not excised, but instead, assayed in vivo, for example, by using agents that can measure the real-time levels of a gene or gene product in the patient's tissue.

In certain embodiments, those patients that have been determined to be at risk of developing metaplasia and are at high degree of risk of developing cancer can then be selected for prophylactic treatment. In exemplary embodiments, the epithelial stem cell crypts that give rise to the metaplasia can be proactively and selectively ablated, such as using techniques described above, before any occurrence of transformed cells or development of esophageal or other cancers.

### E. Screening Methods

In another aspect, the invention provides methods of identifying a compound useful for treating Barrett's esophagus (e.g., esophageal intestinal metaplasia).

In one embodiment, the method generally comprises administering a test compound to a p63 null mouse and determining the amount of epithelial metaplasia in the presence and absence of the test compound, wherein a decrease in the amount of epithelial metaplasia identifies a compound useful for treating esophageal metaplasia.

Suitable p63 null mice include mice with complete germ-line deletion of the p63 gene (see e.g., Yang et al. Nature 1999; 398: 714-8), mice in which the p63 gene has been conditionally deleted in one or more epithelial tissue, and mice in which the cellular levels of p63 protein have been reduced (e.g., by RNAi-mediated gene silencing).

In another embodiment, the method generally comprises administering a test compound to a mouse, wherein the mouse comprises stratified epithelial tissue in which basal cells have been ablated, and determining the amount of epithelial metaplasia in said epithelial tissue in the presence and absence of the test compound, wherein a decrease in the amount of epithelial metaplasia identifies a compound useful for treating esophageal metaplasia.

The basal cells of the mouse stratified epithelial tissue can be ablated using any art-recognized means. In a preferred embodiment, basal cells are ablated using Cre-mediated expression of diphtheria toxin fragment A as described in Ivanova et al. Genesis. 2005; 43:129-35.

The amount of epithelial metaplasia can be determined by any means, including by the examination of pathological specimens obtained from sacrificed mice.

The test compound can be administered to the mice by any route and means that will achieve delivery of the test compound to the requisite location.

In another embodiment, the method generally comprises administering a test compound to a Barrett's esophagus progenitor cell, wherein in the presence and absence of the test compound, wherein a decrease in the viability of the Barrett's esophagus progenitor cell identifies a compound useful for treating esophageal metaplasia. The reduction in viability can be a 50, 60, 70, 75, 80, 85, 90, 95, 96, 97, 98, 99 or 100% reduction in viability. Control cells to show specificity could include esophageal stem cells and gastric cardia stem cells.

### IV. Exemplification

The invention now being generally described, it will be more readily understood by reference to the following examples, which are included merely for purposes of illustration of certain aspects and embodiments of the present invention, and are not intended to limit the invention.

### General Methods

In general, the practice of the present invention employs, unless otherwise indicated, conventional techniques of chemistry, molecular biology, recombinant DNA technology, immunology (especially, e.g., immunoglobulin technology), and animal husbandry. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al, C.S.H.L. Press, Pub. (1999); Current Protocols in Molecular Biology, eds. Ausubel et al., John Wiley & Sons (1992).

### Animal Models

p63−/− mice used in this study were backcrossed 10-12 times on a BALB/c background (Yang et al., 1999 supra). Wild type controls were derived from littermates. To obtain staged embryos, heterozygotes were crossed and the presence of vaginal plugs set the timing at E0.5. The heterozygous DTA-Krt14-Cre strain was generated by crossing the homozygous Gt(ROSA)26Sor<tm1(DTA)Jpmb>/J stain (Ivanova et al. Genesis. 2005; 43:129-35. (Jackson Laboratory) with the homozygous Tg(KRT14-cre/Esr1)20Efu/J (see Vasioukhin et al. Proc Natl Acad Sci USA. 1999; 96:8551-6) (Jackson laboratory). Diphtheria toxin A was transcriptionally activated in basal cells of stratified epithelia via intraperitoneal injection of Tamoxifen in corn oil (100 mg/kg) one to three weeks prior to analysis. Porcine gastroesophageal junctions of three-month-old pigs were obtained from a local abattoir in Strasbourg. Human gastrointestinal junctions were obtained from autopsies at the Brigham and Women's Hospital under IRB approval.

### Expression Microarrays and Bioinformatics

All Cel files were processed using GeneChip Operating Software to calculate probeset intensity values, and probe hybridization ratios were calculated using Affymetrix Expression Console Software to valid sample quality. These intensity values were log 2 transformed and then imported into Partek Genomics Suite 6.5 (beta). A 1-way ANOVA was performed to identify differentially expressed genes. For each analysis, fold-changes and p-values for probesets were calculated. Principal component analysis (PCA) was carried out using all probesets, and heatmaps were generated using sorted datasets based on Euclidean distance and average linkage methods.

Gene expression datasets from normal and Barrett's esophagus were downloaded from the Gene Expression Omnibus (GEO) Genesets of the NCBI (Stairs et al. PLoS One. 2008; 3:e3534). Barrett's metaplasia datasets containing considerable squamous gene expression were excluded from the analysis.

### Histology and Immunofluorescence

Histology, immunohistochemistry, and immunofluorescence were performed using standard techniques. Details on the primary and secondary antibodies employed in these studies are detailed in the Appendix.

### Example 1

### Gene Expression of Barrett's Esophagus Stem Cell Compared to Gastric Cardia Stem Cells

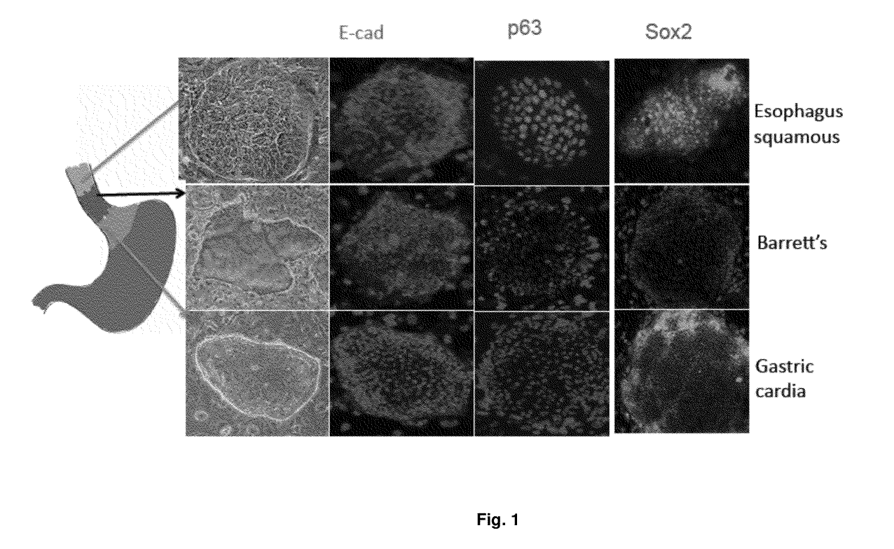

In this study, epithelial stem cells were cloned from distal esophageal squamous tissue, Barrett's and gastric cardia tissue of the same patient. The epithelial stem cells from these three different tissue types were characterized by immunostaining using different markers. These three different tissue types were all found to be E-cadherin positive suggesting that they are all from epithelial tissues. Only esophageal stem cells were p63 positive, which is consistent with its origin of the squamous tissue. Both esophageal and gastric cardia stem cells were SOX2 positive, which is consistent with previous published findings. Importantly, the Barrett's stem cells are SOX2- and p63-negative, suggesting that they represent a distinct population of the cells (FIG. 1).

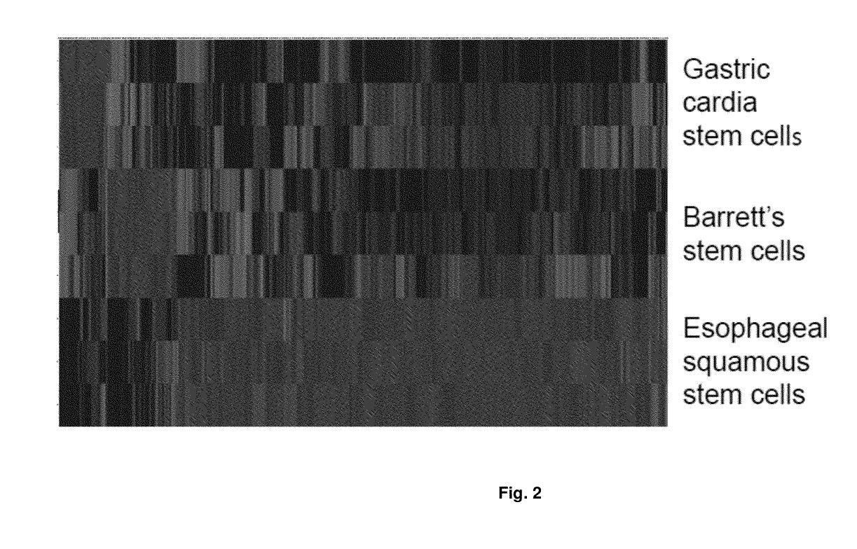

We next compared RNA from three independent stem cell clones of each tissue type (esophagus, Barrett's and gastric cardia) on expression microarray chips. These data revealed distinct gene expression signature of stem cells from different tissues (shown in the heatmap in FIG. 2). Among the differentially expressed genes, 14 cell surface markers were identified that were significantly overexpressed in Barrett's stem cells in comparison with the nearby tissues, such as gastric cardia stem cells. They are listed in Table 3, shown below.

TABLE 3

| Gene | Fold-Change |
|---|---|
| Symbol | (BE vs. Ca) |
| CDH17 | 77.4785 |
| CLRN3 | 32.3754 |
| TM4SF4 | 30.5791 |
| FAM3B | 27.3832 |
| NMUR2 | 26.7482 |
| MUC17 | 19.1791 |
| CEACAM7 | 18.7726 |
| ANXA13 | 13.9535 |
| SLC16A4 | 9.63403 |
| CD44 | 9.3912 |
| NPNT | 8.40884 |
| PLBD1 | 6.54229 |
| TFF3 | 5.66225 |
| REG4 | 5.63931 |

The expression of all of the genes shown in Table 3 is more than 10% of actin expression. One of the genes is CDH17.

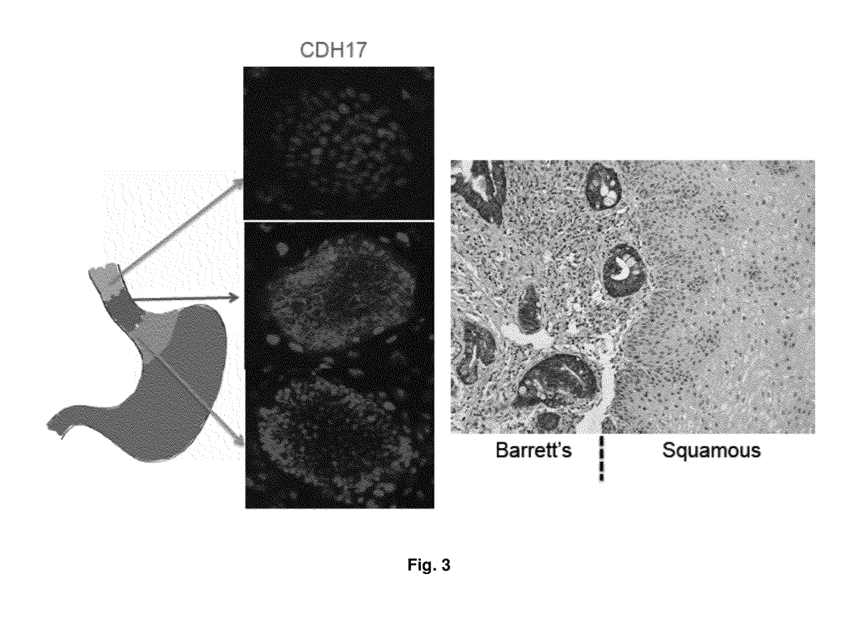

CDH17 expression was validated by immunostaining the cells in culture. This demonstrated that CDH17 protein was specifically expressed in Barrett's stem cells in culture. In addition, CDH17 protein is specifically expressed in human Barrett's esophagus as shown by immunostaining on human BE sections. The CDH17 protein was not detected in esophagus, squamous or gastric cardia (FIG. 3).

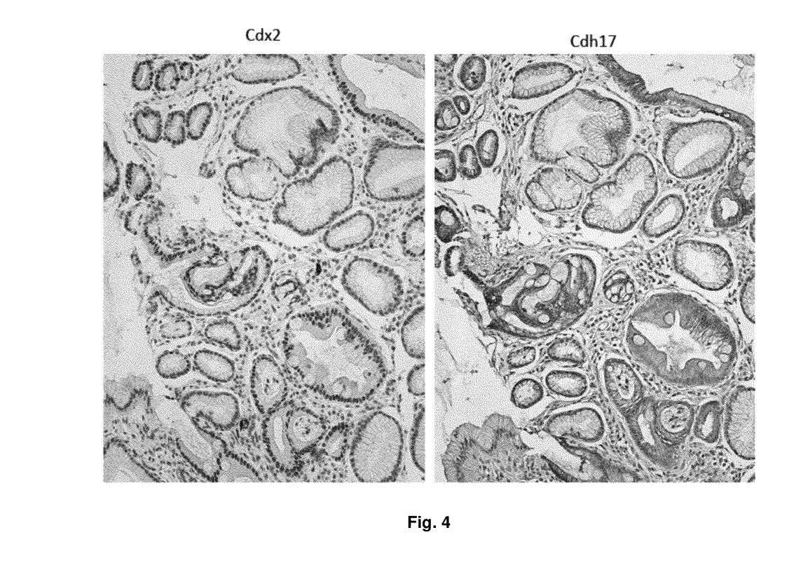

Therefore, CDH17 is a novel and specific marker for both Barrett's stem cells and their differentiated progeny. CDH17 is very different from the other existing Barrett's markers, such as Villin and CDX2, which are only detected in differentiated Barrett's and not the stem cells of Barrett's. This makes CDH17 an attractive molecule for diagnosis of Barrett's esophagus as well as for targeted therapies for BE stem cells. CDH17 was also detected in gastric intestinal metaplasia (FIG. 4). Gastric intestinal metaplasia can also be validated by Villin and Cdx2 staining. The other 14 markers (see Table 3) can also be used to recognize the stem cells of Barrett's esophagus as well as differentiated Barrett's esophagus.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition comprising a clonal population of stem cells isolated from an esophagus of a subject with Barrett's esophagus, wherein the stem cells differentiate into Barrett's epithelium, wherein the stem cells are characterized as having an mRNA profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA are each in the range of 5 to 50 percent of the amount of actin mRNA in the stem cell.

2. The composition of claim 1, wherein the stem cells are characterized as having an mRNA profile wherein the amount of one or more CDH17, CLRN3, TM4SF4, FAM3B, NMUR2, MUC17, CEACAM7, ANXA13, SLC16A4, CD44, NPNT, PLBD1, TFF3 and REG4 mRNA are each at least 10 percent of the amount of actin mRNA in the stem cell.

3. The composition of claim 1, wherein the mRNA is CDH17 mRNA.

4. A purified cell preparation comprising Barrett's esophagus (BE) stem cells, wherein the BE stem cells differentiate into columnar epithelial cells and are characterized as having an mRNA profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA are each in the range of 5 to 50 percent of the amount of actin mRNA in the stem cell.

5. The purified cell preparation of claim 4, wherein the stem cells are characterized as having an mRNA profile wherein the amount of one or more CDH17, CLRN3, TM4SF4, FAM3B, NMUR2, MUC17, CEACAM7, ANXA13, SLC16A4, CD44, NPNT, PLBD1, TFF3 and REG4 mRNA are each at least 10 percent of the amount of actin mRNA in the stem cell.

6. The purified cell preparation of claim 4, wherein the mRNA is CDH17 mRNA.

7. An isolated Barrett's esophagus (BE) stem cell capable of producing columnar epithelial cells, which BE stem cell is characterized as having an mRNA profile wherein the amount of one or more of CDH17, CLRN3, TM4SF4, FAM3B, NMUR2, MUC17, CEACAM7, ANXA13, SLC16A4, CD44, NPNT, PLBD1, TFF3 and REG4 mRNA are each in the range of 5 to 50 percent of the amount of actin mRNA in the stem cell.

8. The isolated Barrett's esophagus (BE) stem cell of claim 7, wherein the stem cell is characterized as having an mRNA profile wherein the amount of one or more CDH17, CLRN3, TM4SF4, FAM3B, NMUR2, MUC17, CEACAM7, ANXA13, SLC16A4, CD44, NPNT, PLBD1, TFF3 and REG4 mRNA are each at least 10 percent of the amount of actin mRNA in the stem cell.

9. The isolated Barrett's esophagus (BE) stem cell of claim 7, wherein the mRNA is CDH17 mRNA.

10. A method of screening for an agent which may be used to treat or prevent the occurrence of Barrett's esophagus, comprising
a) providing BE stem cells;
b) contacting the BE stem cells with the test agent;
c) detecting the ability of the test agent to reduce viability, growth or differentiation of the BE stem cells;
wherein if the test agent reduces the viability, growth or differentiation of the BE stem cells than the test agent may be effective in the treatment or prevention of Barrett's esophagus and wherein the test agent specifically binds to or reduces the expression of one or more of CDH17, CLRN3, TM4SF4, FAM3B, NMUR2, MUC17, CEACAM7, ANXA13, SLC16A4, CD44, NPNT, PLBD1, TFF3 and REG4.

11. The method of claim 10, wherein the mRNA with reduced expression is CDH17.

12. The method of claim 11, wherein the test agent is also contacted with normal cells or tissue of the local alimentary canal, and the differential ability, if any, of the test agent to reduces the viability, growth or differentiation of the normal cells or tissue is compared to that with the BE stem cells.

13. The method of claim 11, wherein the BE stem cells are human BE stem cells.

14. The method of claim 11, wherein the test agent is selected for further drug development if the test agent reduces the viability, growth or ability to differentiation of the BE stem cells is reduced by at least 70%.

15. The method of claim 11, wherein the BE stem cells are provided as a clonal population of cells.

16. The method of claim 11, wherein the test agent is small molecule, carbohydrate, peptide or nucleic acid.

17. The method of claim 11, wherein the test agent specifically binds to a cell surface protein on the clonal population of cells.

18. The method of claim 11, wherein the test agent is an antibody or antibody mimetic.

19. A method of screening for an agent effective in the detection of Barrett's esophagus comprising
a) providing a BE stem cells;
b) contacting the BE stem cells with the test agent;
c) detecting the ability of the test agent to bind to the BE stem cells;
wherein if the test agent binds to the BE stem cells, the test agent may be an agent effective in the detection of Barrett's esophagus wherein the test agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4.

20. The method of claim 19, wherein the test agent specifically binds to CDH17.

21. The method of claim 19, wherein the BE stem cells are human BE stem cells.

22. The method of claim 19, wherein the BE stem cells are provided as a clonal population of cells.

23. The method of claim 19, wherein the test agent is also contacted with normal cells or tissue of the alimentary canal, and the differential ability, if any, of the test agent to bind to the normal cells or tissue is compared to that with the BE stem cells.

24. The method of claim 19, wherein the test agent is an antibody or antibody mimetic.

25. The method of claim 24, wherein the test agent is a monoclonal antibody.

26. A method of detecting the presence of esophageal metaplasia, such as associated with Barrett's esophagus, in a patient comprising
a) providing a detection agent that specifically binds to a BE stem cell relative to normal tissue of the esophagus and (optionally) stomach;
b) administering the detection agent to a patient or contacting the detection agent with a biopsy therefrom; and
c) detecting whether the detection agent binds to BE stem cells in the esophagus of the patient,
wherein the detection agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4.

27. The method of claim 26, wherein the detection agent specifically binds to CDH17.

28. The method of claim 26, wherein the patient is a human.

29. The method of claim 26, wherein the detection step is performed in vitro on a biopsy sample.

30. The method of claim 26, wherein the detection step is performed in vivo.

31. The method of claim 26, wherein the detection agent is an antibody.

32. The method of claim 31, wherein the detection agent is a monoclonal antibody.

33. The method of claim 26, wherein the detection agent is Positron Emission Tomography (PET) imaging agent or magnetic resonance imaging (MRI) contrast agent.

34. The method of claim 26, wherein the detection agent is radioisotope or contrast enhancing isotope, such as ³H, ¹¹C, ¹⁷⁷Lu, ¹¹¹Indium, ⁶⁷Cu, ^{99m}Tc, ¹²⁴I, ¹²⁵I, ¹³¹I and ⁸⁹Zr.

35. The method of claim 26, wherein the detection agent is detected in the patient by Single Photon Emission Computed Tomography (SPECT), Positron Emission Tomography (PET), Magnetic Resonance Imaging (MRI), Fluorescent Imaging, or Near-infrared (NIR) Emission Spectroscopy.

36. A method for treating or preventing Barrett's esophagus and/or esophageal metaplasia in a subject in need thereof comprising administering to the subject an effective amount of an therapeutic agent that is cytotoxic or cytostatic for Barrett's Esophagus (BE) stem cells in the esophagus of the subject, or inhibits differentiation of the BE stem cells to columnar epithelium, wherein the therapeutic agent specifically binds to or reduces the expression of one or more of CDH17, CLRN3, TM4SF4, FAM3B, NMUR2, MUC17, CEACAM7, ANXA13, SLC16A4, CD44, NPNT, PLBD1, TFF3 and REG4.

37. The method of claim 36, wherein the therapeutic agent specifically binds to or reduces the expression of CDH17.

38. The method of claim 36, wherein the subject is a mammal.

39. The method of claim 38, wherein the mammal is a human.

40. The method of claim 36, wherein the differentiation of the BE stem cells to columnar epithelium is reduced by 70, 80, 90, 95, 96, 97, 98, 99 or 100% from treatment with the therapeutic agent.

41. The method of claim 36, wherein the therapeutic agent is an antibody or antibody mimetic.

42. The method of claim 41, wherein the therapeutic agent is a monoclonal antibody.

43. The method of claim 41 or 42, wherein the antibody or antibody mimetic is conjugated to a cytotoxic or cytostatic moiety.

44. The method of claim 36, wherein the therapeutic agent is a nucleic acid or nucleic acid analog.

45. The method of claim 44, wherein the therapeutic agent is an RNAi or antisense composition.

46. The method of claim 45, wherein the RNAi or antisense composition reduces the level of expression of a gene selected from the group consisting of CDH17, CLRN3, TM4SF4, FAM3B, NMUR2, MUC17, CEACAM7, ANXA13, SLC16A4, CD44, NPNT, PLBD1, TFF3 and REG4.

47. The method of claim 46, wherein the gene is CDH17.

48. A method of detecting the presence of Barrett's esophagus (BE) stem cells, in a patient comprising
a) providing a detection agent that specifically binds to a BE stem cell relative to normal tissue of the esophagus and (optionally) stomach;
b) administering the detection agent to a patient or contacting the detection agent with a biopsy therefrom; and
c) detecting whether the detection agent binds to BE stem cells in the esophagus of the patient,
wherein the detection agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4.

49. The method of claim 48, wherein the detection agent specifically binds to CDH17.

50. The method of claim 48, wherein the patient is a human.

51. The method of claim 48, wherein the detection step is performed in vitro on a biopsy sample.

52. The method of claim 48, wherein the detection step is performed in vivo.

53. The method of claim 48, wherein the detection agent is an antibody.

54. The method of claim 53, wherein the detection agent is a monoclonal antibody.

55. The method of claim 48, wherein the detection agent is Positron Emission Tomography (PET) imaging agent or magnetic resonance imaging (MRI) contrast agent.

56. The method of claim 48, wherein the detection agent is radioisotope or contrast enhancing isotope, such as ³H, ¹¹C, ¹⁷⁷Lu, ¹¹¹Indium, ⁶⁷Cu, ^{99m}Tc, ¹²⁴I, ¹²⁵I, ¹³¹I and ⁸⁹Zr.

57. The method of claim 48, wherein the detection agent is detected in the patient by Single Photon Emission Computed Tomography (SPECT), Positron Emission Tomography (PET), Magnetic Resonance Imaging (MRI), Fluorescent Imaging, or Near-infrared (NIR) Emission Spectroscopy.

58. A method of detecting the likelihood of the presence of intestinal metaplasia (IM), in a patient comprising
a) providing a detection agent that specifically binds to CDH17;
b) administering the detection agent to a patient or contacting the detection agent with a biopsy therefrom; and
c) detecting whether the detection agent binds to the cells in the biopsy of the patient,
wherein if the detection agent binds to the cells in the biopsy of the patient the IM is more likely to be present in the patient.

59. The method of claim 58, further comprising the steps of
d) providing a detection agent that specifically binds to Villin;
e) administering the detection agent to a patient or contacting the detection agent with a biopsy therefrom; and
f) detecting whether the detection agent binds to the cells in the biopsy of the patient.

60. The method of claim 58, further comprising the steps of
d) providing a detection agent that specifically binds to CDX2;
e) administering the detection agent to a patient or contacting the detection agent with a biopsy therefrom; and
f) detecting whether the detection agent binds to the cells in the biopsy of the patient.

61. The method of claim 58, wherein the intestinal metaplasia comprises a cell type selected from the group consisting of Barrett's esophagus stem cells, gastric adenocarcinoma precursors and pancreatic adenocarcinoma precursors.

62. The method of claim 58, wherein the intestinal metaplasia is selected from the group consisting of Barrett's esophagus, gastric intestinal metaplasia and intraepithelial pancreatic mucinous metaplasia.

63. The method of claim 58, wherein the patient is a human.

64. The method of claim 58, wherein the detection step is performed in vitro on a biopsy sample.

65. The method of claim 58, wherein the detection step is performed in vivo.

66. The method of claim 58, wherein the detection agent is an antibody.

67. The method of claim 66, wherein the detection agent is a monoclonal antibody.

68. The method of claim 58, wherein the detection agent is Positron Emission Tomography (PET) imaging agent or magnetic resonance imaging (MRI) contrast agent.

69. The method of claim 58, wherein the detection agent is radioisotope or contrast enhancing isotope, such as ³H, ¹¹C, ¹⁷⁷Lu, ¹¹¹Indium, ⁶⁷Cu, ^{99m}Tc, ¹²⁴I, ¹²⁵I, ¹³¹I and ⁸⁹Zr.

70. The method of claim 58, wherein the detection agent is detected in the patient by Single Photon Emission Computed Tomography (SPECT), Positron Emission Tomography (PET), Magnetic Resonance Imaging (MRI), Fluorescent Imaging, or Near-infrared (NIR) Emission Spectroscopy.

71. A composition comprising a binding agent that specifically binds to a protein selected from the group consisting of CDH17, CLRN3, TM4SF4, FAM3B, NMUR2, MUC17, CEACAM7, ANXA13, SLC16A4, CD44, NPNT, PLBD1, TFF3 and REG4 attached to an imaging agent.

72. The composition of claim 71, wherein the protein is CDH17.

73. The composition of claim 71, wherein the binding agent is covalently attached to the imaging agent.

74. The composition of claim 71, wherein the binding agent is non-covalently attached to the imaging agent.

75. The composition of claim 71, wherein the imaging agent is selected from the group consisting of optical coherence tomography (OCT) detection/contrast agents, positron emission tomography (PET) detection/contrast agents, magnetic resonance imaging (MRI) detection/contrast agents, ultrasound detection/contrast agents, X-ray detection/contrast agents and single-photon emission computed tomography (SPECT) detection/contrast agents.

76. The composition of claim 75, wherein the OCT detection/contrast agents are selected from the group consisting of near-infrared dyes, polypyrrole nanoparticles, optical detection/contrast agents and engineered microsphere contrast agents.

77. The composition of claim 75, wherein the PET detection/contrast agents are selected from the group consisting of ¹⁸F-fluoride, 3′-deoxy-3′-[¹⁸F]fluorothymidine, ¹⁸F-fluoromisonidazole, gallium, technetium-99m, thallium, oxygen, nitrogen, iron, carbon, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc.

78. The composition of claim 75, wherein the MRI detection/contrast agents are selected from the group consisting of ferro, antiferro, ferrimagnetic or superparamagnetic material, ferrite with spinel structure, ferrite with a magnetoplumbite structure other hexagonal ferrite structures, paramagnetic ions, comprise a paramagnetic contrast agent, a super paramagnetic contrast agent, a diamagnetic agent and combinations thereof.

79. The composition of claim 75, wherein the ultrasound detection/contrast agents are selected from the group consisting of shell encapsulated gas bubbles; shell encapsulated droplets; and nanoparticles.

80. The composition of claim 75, wherein the X-ray detection/contrast agents are selected from the group consisting of iodinated contrast-enhancing units; barium sulfate-based contrast-enhancing units; metal ion chelates; boron clusters with a high proportion of iodine; iodinated polysaccharides, polymeric triiodobenzenes; particles from iodinated compounds displaying low water solubility; liposomes containing iodinated compounds; and iodinated.

81. The composition of claim 75, wherein the SPECT detection/contrast agents are selected from the group consisting of ⁹⁹mTc, ¹²³I, ¹³¹I, ⁶⁷Cu, ¹¹¹In, and ²⁰¹Tl.

82. The composition of claim 75, wherein the binding agent is selected from the group consisting of antibodies, aptamers, peptides, cell surface receptor ligands, and small molecules.

83. A method of screening for an agent which may be used to treat or prevent the occurrence of Barrett's esophagus, comprising
a) providing BE stem cells;
b) providing one or more of esophageal and gastric cardia stem cells;
c) contacting the BE stem cells and one or more of esophageal and gastric cardia stem cells with the test agent; and
d) detecting the ability of the test agent to reduce viability, growth or differentiation of the BE stem cells relative to one or more of esophageal and gastric cardia stem cells;
wherein if the test agent reduces the viability, growth or differentiation of the BE stem cells relative to one or more of esophageal and gastric cardia stem cells than the test agent may be effective in the treatment or prevention of Barrett's esophagus.

84. The method of claim 83, wherein the test agent specifically binds to or reduces the mRNA expression of one or more of CDH17, CLRN3, TM4SF4, FAM3B, NMUR2, MUC17, CEACAM7, ANXA13, SLC16A4, CD44, NPNT, PLBD1, TFF3 and REG4.

85. The method of claim 84, wherein the test agent specifically binds to CDH17.

86. The method of claim 84, wherein the mRNA with reduced expression is CDH17.

87. The method of claim 84, wherein the test agent is also contacted with normal cells or tissue of the local alimentary canal, and the differential ability, if any, of the test agent to reduces the viability, growth or differentiation of the normal cells or tissue is compared to that with the BE stem cells.

88. The method of claim 84, wherein the BE stem cells are human BE stem cells.

89. The method of claim 84, wherein the test agent is selected for further drug development if the test agent reduces the viability, growth or ability to differentiation of the BE stem cells is reduced by at least 70%.

90. The method of claim 84, wherein the BE stem cells are provided as a clonal population of cells.

91. The method of claim 84, wherein the test agent is a small molecule, carbohydrate, peptide or nucleic acid.

92. The method of claim 84, wherein the test agent specifically binds to a cell surface protein on the clonal population of cells.

93. The method of claim 84, wherein the test agent is an antibody or antibody mimetic.

94. A method of screening for an agent effective in the detection of Barrett's esophagus comprising
a) providing a BE stem cells;
b) providing one or more of esophageal and gastric cardia stem cells;
c) contacting the BE stem cells and one or more of esophageal and gastric cardia stem cells with the test agent; and
d) detecting the ability of the test agent to bind to the BE stem cells and one or more of esophageal and gastric cardia stem cells;
wherein if the test agent binds to the BE stem cells with greater affinity than it binds to one or more of esophageal and gastric cardia stem cells, the test agent may be an agent effective in the detection of Barrett's esophagus.

95. The method of claim 94, wherein the test agent specifically binds to one or more of CDH17, CLRN3, TM4SF4, FAM3B, NMUR2, MUC17, CEACAM7, ANXA13, SLC16A4, CD44, NPNT, PLBD1, TFF3 and REG4.

96. The method of claim 95, wherein the test agent specifically binds to CDH17.

97. The method of claim 94, wherein the BE stem cells are human BE stem cells.

98. The method of claim 94, wherein the BE stem cells are provided as a clonal population of cells.

99. The method of claim 97, wherein the test agent is also contacted with normal cells or tissue of the alimentary canal, and the differential ability, if any, of the test agent to bind to the normal cells or tissue is compared to that with the BE stem cells.

100. The method of claim 94, wherein the test agent is an antibody or antibody mimetic.

101. The method of claim 100, wherein the test agent is a monoclonal antibody.
